(19) United States
(12) Patent Application Publication
(10) Pub. No.: US 2025/0213586 A1
(43) Pub. Date: Jul. 3, 2025

(54) STAT6 INHIBITORS AND USES THEREOF

(71) Applicant: Kymera Therapeutics, Inc., Watertown, MA (US)

(72) Inventors: Huijun Dong, Watertown, MA (US); Bin Yang, Watertown, MA (US); Bruce C. Follows, Watertown, MA (US); Xiao Zhu, Watertown, MA (US); Matthew M. Weiss, Watertown, MA (US); Xiaozhang Zheng, Watertown, MA (US); Xin Huang, Watertown, MA (US); Lijing Su, Watertown, MA (US); Nello Mainolfi, Watertown, MA (US); Thijs Beuming, Cambridge, MA (US); Xue Fei, Watertown, MA (US); Philip Collier, Watertown, MA (US); Yi Zhang, Watertown, MA (US)

(21) Appl. No.: 19/008,194

(22) Filed: Jan. 2, 2025

Related U.S. Application Data
(60) Provisional application No. 63/649,869, filed on May 20, 2024.
(60) Provisional application No. 63/617,310, filed on Jan. 3, 2024.

(51) Int. Cl. A61K 31/55; A61K 31/405; A61K 31/4192; A61K 31/427; A61K 31/4439; A61K 31/444; A61K 31/4709; A61K 31/496; A61K 31/501; A61K 31/538; C07D 209/18; C07D 211/34; C07D 401/04; C07D 401/06; C07D 401/10; C07D 401/14; C07D 403/04; C07D 403/12; C07D 403/14; C07D 405/12; C07D 405/14; C07D 409/14; C07D 413/06; C07D 413/14; C07D 417/04; C07D 471/04; C07D 487/04

(52) U.S. Cl. CPC A61K 31/55; A61K 31/405; A61K 31/4192; A61K 31/427; A61K 31/4439; A61K 31/444; A61K 31/4709; A61K 31/496; A61K 31/501; A61K 31/538; C07D 209/18; C07D 211/34; C07D 401/04; C07D 401/06; C07D 401/10; C07D 401/14; C07D 403/04; C07D 403/12; C07D 403/14; C07D 405/12; C07D 405/14; C07D 409/14; C07D 413/06; C07D 413/14; C07D 417/04; C07D 471/04; C07D 487/04

(57) ABSTRACT

The present invention provides compounds, compositions, and methods of using the same.

### CROSS-REFERENCE TO RELATED APPLICATIONS

This application claims the benefit of priority to U.S. Provisional Application No. 63/617,310, filed Jan. 3, 2024, and U.S. Provisional Application No. 63/649,869, filed May 20, 2024.

### TECHNICAL FIELD OF THE INVENTION

The present invention relates to compounds and methods useful for the inhibition of signal transducer and activator of transcription 6 (“STAT6”). The invention also provides pharmaceutically acceptable compositions comprising compounds of the present invention and methods of using said compositions in the treatment of various disorders.

### BACKGROUND OF THE INVENTION

Signal transducer and activator of transcription 6 (STAT6 or Interleukin-4-Stat/IL4-STAT) is an undruggable transcription factor belonging to the structurally conserved Signal Transducer and Activator of Transcription (STAT) family of proteins (STAT1 through STAT6). Activation of STAT6, like other STAT proteins, is triggered upon binding of hormones, immunomodulatory cytokines or growth factors to specific receptors on the cell surface. Once activated, the phosphorylation of a C-terminal tyrosine residue occurs, leading to translocation and transmission of signals from the cytosol to the nucleus, resulting in activation of gene expression.

STAT6 is implicated in driving Type 2 immunity, allergies. It may participate in IL-4/IL-13-mediated allergic reaction, and play a vital role in the differentiation of T-helper type 2 (Th2) cells (Hebenstreit et al. “Signaling mechanisms, interaction partners, and target genes of STAT6.” Cytokine & growthfactor reviews 17.3 (2006): 173-188; Chapoval et al. “Regulation of the T helper cell type 2 (Th2)/T regulatory cell (Treg) balance by IL-4 and STAT6.” Journal of leukocyte biology 87.6 (2010): 1011-1018). STAT6 is a key node primarily activated in the Janus Kinase (JAK) pathway by inflammatory cytokines, interleukin-4 (IL4) and interleukin-13 (IL13) and their cognate receptors, which are produced by Th2 cells, mast cells and basophils. Human STAT6 mutations have been associated with severe allergies such as asthma and eczema (Goenka and Kaplan. “Transcriptional regulation by STAT6.” Immunologic research 50.1 (2011): 87-96.). There is a need to discover and develop STAT6 drugs, for example to treat allergic/inflammatory diseases and cancers (Glosson et al. “Wheezing and itching: The requirement for STAT proteins in allergic inflammation.” Jak-Stat 1.1 (2012): 3-15; Loh et al. “Signal transducer and activator of transcription (STATs) proteins in cancer and inflammation: functions and therapeutic implication.” Frontiers in oncology 9 (2019): 48). Accordingly, there remains a need to find STAT6 inhibitors useful as therapeutic agents.

### SUMMARY OF THE INVENTION

It has now been found that compounds of this invention, and pharmaceutically acceptable compositions thereof, are effective as inhibitors of STAT6 protein.

In some embodiments, the present disclosure provides inhibitor compounds of formula I-a:

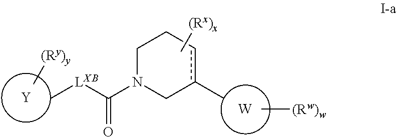

or a pharmaceutically acceptable salt thereof, wherein each variable is as defined and described herein.

In some embodiments, the present disclosure provides inhibitor compounds of formula I-b:

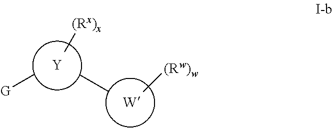

or a pharmaceutically acceptable salt thereof, wherein each variable is as defined and described herein.

Inhibitor compounds described herein, and pharmaceutically acceptable compositions thereof, are useful for treating a variety of diseases, disorders or conditions, associated with regulation of signaling pathways implicating STAT6 protein. Such diseases, disorders, or conditions include those described herein.

Inhibitor compounds described herein are also useful for the study of STAT6 protein in biological and pathological phenomena; the study of intracellular signal transduction pathways occurring in bodily tissues; and the comparative evaluation of new STAT6 inhibitors or other regulators of cell cycling, metastasis, angiogenesis, and immune cell evasion, in vitro or in vivo.

### DETAILED DESCRIPTION OF CERTAIN EMBODIMENTS

### 1. General Description of Certain Embodiments of the Invention

Inhibitor compounds described herein, and compositions thereof, are useful as inhibitors of STAT6 protein which includes phosphorylated or activated STAT6 protein, e.g., pSTAT6. In some embodiments, a provided inhibitor compound inhibits and/or modulates STAT6, pSTAT6, or STAT6 and pSTAT6.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclic ring may be a 5-12 membered bicyclic, bridged bicyclic, or spirocyclic ring. A carbocyclic ring may include one or more oxo (═O) or thioxo (═S) substituent. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

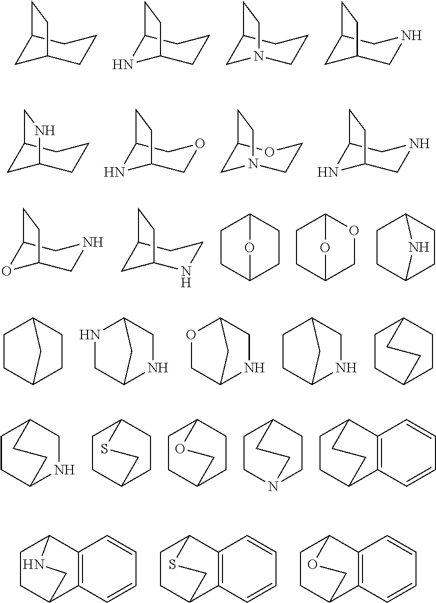

The term “lower alkyl” refers to a C₁₋₄ straight or branched alkyl group. Exemplary lower alkyl groups are methyl, ethyl, propyl, isopropyl, butyl, isobutyl, and tert-butyl.

The term “haloalkyl” refers to a C₁₋₆ straight or branched alkyl group that is substituted with one or more halogen atoms and “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

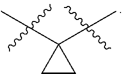 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “arylenyl” refers to bivalent aryl groups (e.g., phenylenyl).

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic, bridged bicyclic, or spirocyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “heteroarylenyl” refers to bivalent heteroaryl groups (e.g., pyridylenyl).

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. In some embodiments, a heterocyclic ring may be a 5-12 membered bicyclic, bridged bicyclic, or spirocyclic ring. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄S R^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)₀₋₄P(O)R^{∘}₂; —(CH₂)_{0- 4}OP(O)R^{∘}₂; —(CH₂)₀₋₄OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromatographic purification are contemplated herein (e.g., diammonium salts) and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the term “provided compound” refers to any genus, subgenus, and/or species set forth herein.

The terms “measurable affinity” and “measurably inhibit,” as used herein, means a measurable change in STAT6 protein activity between a sample comprising a compound of the present invention, or composition thereof, and STAT6 protein, and an equivalent sample comprising STAT6 protein, in the absence of said compound, or composition thereof.

As used herein, the term “reference” describes a standard or control relative to which a comparison is performed. In some embodiments, a “reference” sample or subject is one that is sufficiently similar to a particular sample or subject of interest to permit a relevant comparison. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, an “inhibitor compound” is a compound that binds a protein of interest. In some embodiments, an inhibitor compound binds a protein of interest and decreases its activity. In some embodiments, binding of an inhibitor compound to a protein of interest does not result in degradation of the protein of interest. In another embodiment, an inhibitor compound does not bind an E3 ligase in a manner that results in degradation of the protein of interest. In some embodiments, an inhibitor compound binds STAT6. In some embodiments, an inhibitor compound binds STAT6 and decreases its activity. In some embodiments, binding of an inhibitor compound to STAT6 does not result in degradation of STAT6. In another embodiment, an inhibitor compound does not bind an E3 ligase in a manner that results in degradation of the STAT6. In some embodiments, an inhibitor compound binds a protein of interest (e.g., STAT6) but does not bind an E3 ligase. In some embodiments, an E3 ligase is selected from a cereblon E3 ubiquitin ligase, a VHL E3 ubiquitin ligase, a DCAF E3 ubiquitin ligase, (e.g., a DCAF1 E3 ubiquitin ligase, a DCAF15 E3 ubiquitin ligase, or a DCAF16 E3 ubiquitin ligase), an IAP E3 ubiquitin ligase, an MDM2 E3 ligase, or a DC2 E3 ubiquitin ligase. It will be understood that, throughout this disclosure, reference to a “compound” or a “provided compound” refers to an inhibitor compound as defined above.

In some embodiments, an E3 ligase is a cereblon E3 ubiquitin ligase. In some embodiments, the inhibitor compound does not comprise a structure of formula I-aa′:

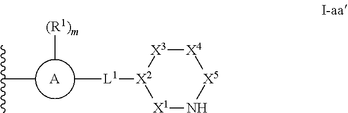

or a pharmaceutically acceptable salt thereof, wherein:
X¹ and X⁵ are independently a covalent bond, —CR₂—, —SO₂—, —S(O)—, —P(O)R—, —P(O)OR—, —P(O)N(R)₂—, —C(O)—, —C(S)—, or

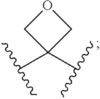 [SMILES: *C1(*)COC1]

X² is N, C—R^{B}, Si—R^{B}, or P═O;
X³ and X⁴ are independently a covalent bond, —CR₂—, —CF₂—, —O—, —S—, or
   X³—X⁴ is —CR═CR—;
each R¹ is independently —H, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R; or:
   two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur;
each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur.

In some embodiments, the inhibitor compound does not comprise

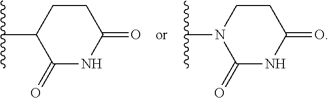 [SMILES: *C1CCC(=O)NC1=O ; *N1CCC(=O)NC1=O]

In some embodiments, an E3 ligase is a VHL E3 ubiquitin ligase. In some embodiments, the inhibitor compound does not comprise a structure of formula I-ccc-3′:

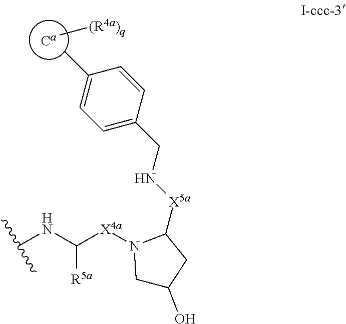

or a pharmaceutically acceptable salt thereof, wherein:
each of X^{4a} and X^{5a} is independently a bivalent moiety selected from —CH₂—, —C(O)—, —C(S)—, or

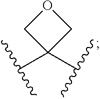 [SMILES: *C1(*)COC1]

each R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R; or:
R^{5a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, a 5-6 membered heteroaryl ring having 1-4 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
Ring C^{a} is a selected from 6-membered aryl containing 0-2 nitrogen atoms or a 5-membered heteroaryl with 1-3 heteroatoms independently selected from nitrogen, oxygen or sulfur;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, the inhibitor compound does not comprise

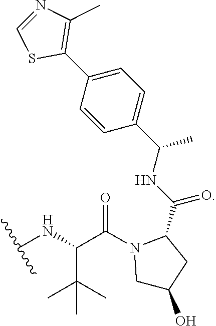 [SMILES: *N[C@H](C(=O)N1C[C@H](O)C[C@H]1C(=O)N[C@@H](C)c1ccc(-c2scnc2C)cc1)C(C)(C)C]

In some embodiments, an E3 ligase is an MDM2 E3 ubiquitin ligase. In some embodiments, the inhibitor compound does not comprise a structure of I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, or I-aaa-18 respectively:

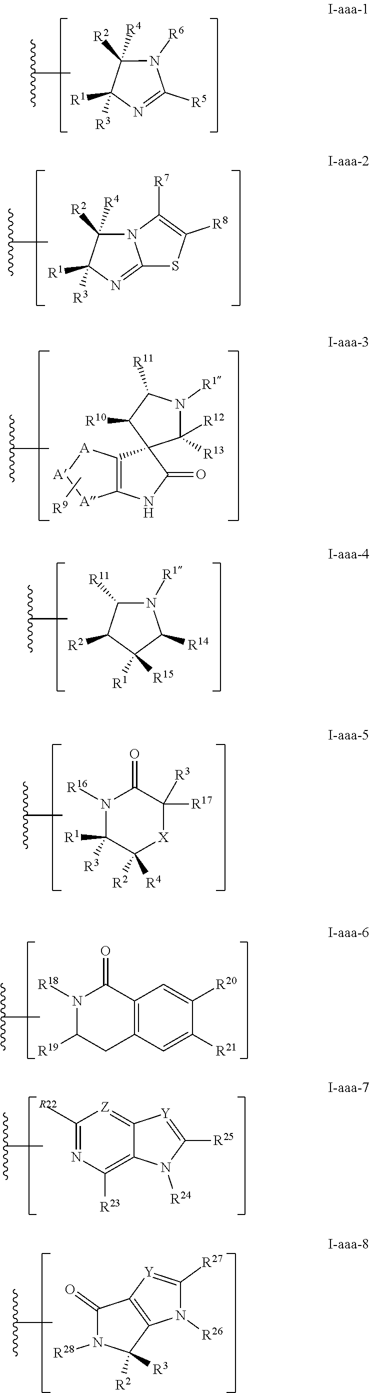

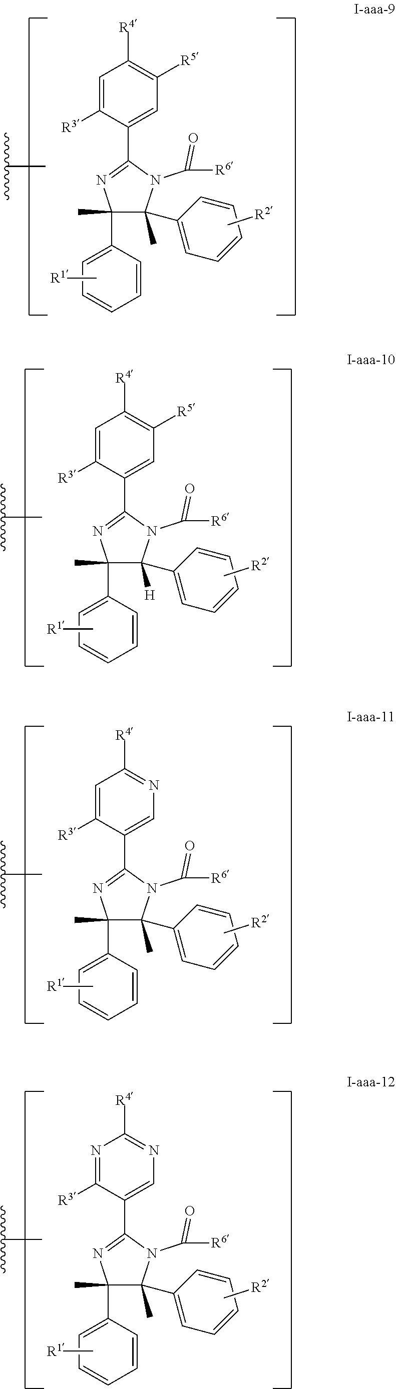

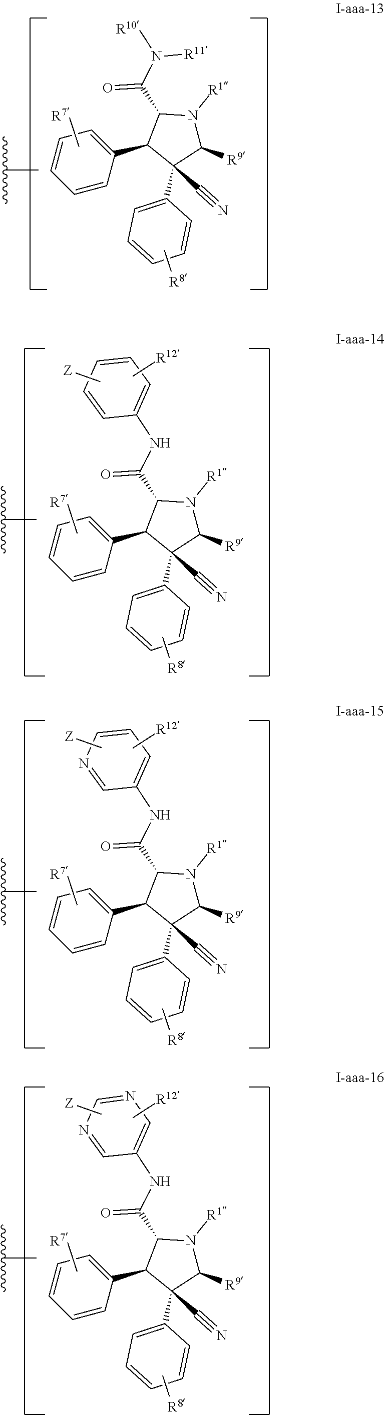

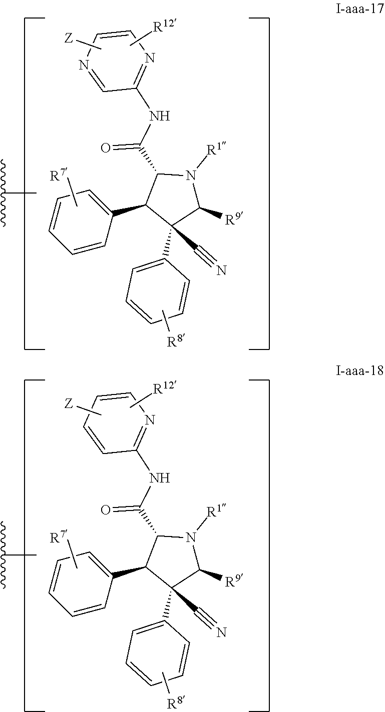

or a pharmaceutically acceptable salt thereof, wherein:
X is selected from —CR₂—, —O—, —S—, —S(O)—, —S(O)₂—, and —NR—;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom from which they are attached, independently selected from nitrogen, oxygen, and sulfur.
Y and Z are independently selected from —CR═ and —N═;
Ring W is fused ring selected from benzo and a 5-6 membered heteroaryl with 1-4 heteroatoms independently selected from nitrogen, oxygen, and sulfur;
R¹ and R² are independently an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R³ and R⁴ are independently selected from hydrogen and C₁₋₆ alkyl;
R⁵ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R⁶ is selected from hydrogen, —C(O)R, —C(O)OR, and —C(O)NR₂;
R⁷ is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁸ is selected from —C(O)R and R^{A};
R⁹ is a mono-, bis-, or tri-substituent on Ring W, wherein each of the substituents are independently selected from halogen and an optionally substituted C₁₋₆ aliphatic;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R¹¹ is —C(O)OR or —C(O)NR₂;
R¹² and R¹³ are independently selected from hydrogen and R^{A}, or:
   R¹² and R¹³ are optionally taken together with their intervening atoms to form an optionally substituted 3-8 membered saturated, partially unsaturated, carbocyclic or heterocyclic ring having 1-3 heteroatoms independently selected from nitrogen, oxygen, and sulfur;
R¹⁴ is R^{A};
R¹⁵ is —CN;
R¹⁶ is selected from R^{A}, —OR, —(CR₂)₀₋₆—C(O)R, —(CR₂)₀₋₆—C(O)OR, —(CR₂)₀₋₆—C(O)NR₂, —(CR₂)₀₋₆—S(O)₂R, —(CR₂)₀₋₆—N(R)S(O)₂R, —(CR₂)₀₋₆—S(O)₂NR₂;
R¹⁷ is selected from —(CR₂)₀₋₆—C(O)NR₂;
R¹⁸ and R¹⁹ are independently selected from hydrogen and R^{A};
R²⁰ and R²¹ are independently selected from hydrogen, R^{A}, halogen, and —OR, or:
   R²⁰ and R²¹ are optionally taken together with their intervening atoms to form a fused 5-7 membered partially unsaturated carbocyclic or heterocyclic ring having 1-3 heteroatoms independently selected from nitrogen, oxygen, and sulfur, or a fused 5-6 membered heteroaryl ring having 1-3 heteroatoms independently selected from nitrogen, oxygen, and sulfur;
R²², R²³, R²⁵, and R²⁷ are independently selected from hydrogen, R^{A}, halogen, —C(O)R, —C(O)OR, —C(O)NR₂, —NR₂, —OR, —S(O)R, —S(O)₂R, —S(O)₂NR₂;
R²⁴, R²⁶, and R²⁸ are independently selected from hydrogen, R^{A}, —C(O)R, —C(O)OR, —C(O)NR₂, —S(O)R, —S(O)₂R, and —S(O)₂NR₂;
R^{1′} and R^{2′} are independently selected from halogen, —C≡CR, —CN, —CF₃, and —NO₂;
R^{3′} is —OR;
R^{4′}, R^{5′}, R^{6′} are independently selected from hydrogen, halogen, R^{A}, —CN, —CF₃, —NR₂, —OR, —SR, and —S(O)₂R;
R^{7′} is a mono-, bis-, or tri-substituent, wherein each of the substituents are independently selected from halogen;
R^{8′} is a mono-, bis-, or tri-substituent, wherein each of the substituents are independently selected from hydrogen, halogen, R^{A}, —CN, —C≡CR, —NO₂, and —OR;
R^{9′} is R^{A};
Z¹ is selected from hydrogen, halogen, and —OR;
R^{10′} and R^{11′} are independently selected from hydrogen and R^{A};
R^{12′} is selected from —C(O)R, —C(O)OR, —C(O)NR₂, —OR, —S(O)₂R, —S(O)₂NR₂, and —S(O)R; and
R^{1″} is selected from hydrogen and R^{A}.

In some embodiments, an E3 ligase is a DCAF E3 ubiquitin ligase (e.g., DCAF1 E3 ubiquitin ligase). In some embodiments, the inhibitor compound does not comprise a structure of formula I-b-a or I-b-b:

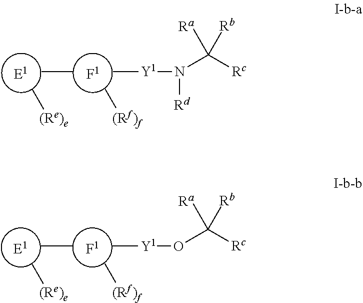

or a pharmaceutically acceptable salt thereof, wherein:
Ring E¹ is phenyl, naphthyl, a 4-9 membered partially unsaturated monocyclic, bicyclic, or bridged bicyclic carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring F¹ is a 5-membered monocyclic heteroarylenyl with 1-3 heteroatoms independently selected from nitrogen, oxygen and sulfur.
Y¹ is a C₁₋₃ hydrocarbon chain wherein each methylene is optionally replaced with —CR₂—, —CR(OR)—, —C(O)—, —C(NR)—, —C(NOR)—, —S(O)—, or —S(O)₂—; or —C(OR)═ in formula I-b-a where R^{d} is absent;
R^{a} is hydrogen, an optionally substituted C₁₋₆ aliphatic, or

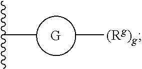

Ring G is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur;
R^{b} is hydrogen, an optionally substituted C₁₋₆ aliphatic, phenyl, or a 5-6 membered heteroaryl with 1-4 heteroatoms independently selected from nitrogen, oxygen and sulfur, or:
   R^{a} and R^{b} are taken together with their intervening atoms to form an optionally substituted 9-10 membered saturated or partially unsaturated bicyclic ring having 0-3 heteroatoms independently selected from nitrogen, oxygen, and sulfur, or:
   when Y¹ is —C(NR)—, R is taken together with R of —C(NR)— with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{c} is —CO₂R, —CONR₂, —CR₂CF₂R, —CR₂CONR₂, —CR₂C(O)R, —CR₂CO₂R, —CR₂NR₂, —CR₂OR, —CR₂SO₂NR₂, —CR₂S(O)R, —CR₂SO₂R, —CR₂S(O)(NR)R, —CR₂CN, —CR₂CR₂NR₂, —CR₂CR₂OR, —CR₂CR═NOR, —CR₂CR(OR)CR₂OR, or an optionally substituted group selected from phenyl; a 4-7 membered saturated or partially unsaturated carbocyclyl or heterocyclyl with 1-3 heteroatoms independently selected from nitrogen, oxygen, and sulfur; a 5-9 membered monocyclic or bicyclic heteroaryl with 1-4 heteroatoms independently selected from nitrogen, oxygen and sulfur; or:
   —(CR₂)₁₋₂—X^{a}, wherein X^{a} is halogen or an optionally substituted ring selected from phenyl; a 4-7 membered saturated or partially unsaturated carbocyclyl or heterocyclyl with 1-3 heteroatoms independently selected from nitrogen, oxygen, and sulfur; and a 5-9 membered monocyclic or bicyclic heteroaryl with 1-4 heteroatoms independently selected from nitrogen, oxygen and sulfur; or:
   R^{b} and R^{c} are taken together with their intervening atoms to form an optionally substituted 4-6 membered saturated or partially unsaturated carbocyclyl or heterocyclyl with 1-3 heteroatoms independently selected from nitrogen, oxygen, and sulfur; or:
   R^{a} is absent and R and R^{c} are taken together with their intervening atoms to form an optionally substituted phenyl; or:
   when Y¹ is —C(OR)═, R^{c} is taken together with R of —C(OR)═ with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{d} is hydrogen or an optionally substituted C₁₋₆ aliphatic, or:
   when R^{c} is —CR₂CONR₂, R^{d} is taken together with a single R of —CR₂CONR₂ with their intervening atoms to form a 5-7 membered saturated or partially unsaturated heterocyclyl with 0-3 heteroatoms, in addition to the nitrogen atom to which R^{d} is attached, independently selected from nitrogen, oxygen, and sulfur;
R^{e}, R^{f}, and R^{g} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —C(NOR)R, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, naphthyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form an optionally substituted 3-7 membered saturated or partially unsaturated ring having 0-3 heteroatoms, in addition to the atom to which they are attached, independently selected from nitrogen, oxygen, and sulfur; and
each of e, f, and g are independently 0, 1, 2, 3, or 4.

In some embodiments, the inhibitor compound does not comprise a structure of formula I-b-c:

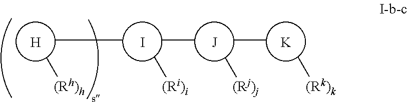

or a pharmaceutically acceptable salt thereof, wherein:
Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
Ring I is phenylenyl or a 5-10 membered monocyclic or bicyclic heteroarylenyl with 1-4 heteroatoms independently selected from nitrogen, oxygen and sulfur;
Ring J is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
Ring K is phenyl, naphthyl, or a 5-13 membered monocyclic, bicyclic, or tricyclic heteroarylenyl with 1-5 heteroatoms independently selected from nitrogen, oxygen and sulfur;
R^{h}, Rⁱ, R^{j}, and R^{k} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂, or:
   an Rⁱ group on Ring I and an R^{j} group or Ring J are optionally taken together with their intervening atoms to form a 5-8 membered saturated, partially unsaturated, or aromatic ring having 0-3 heteroatoms independently selected from nitrogen, oxygen, and sulfur, or:
each of X¹ and X² are independently a covalent bond, spiro-fusion between the two rings that X¹ or X² connect, or a bivalent, saturated or unsaturated, straight or branched C₁_₆ hydrocarbon chain, wherein 0-4 methylene units of X¹ and X² are independently replaced by —CR₂—, —CR(OR)—, —CRF—, —CF₂—, —C(NR)—, —C(O)—, —O—, —N(R)—, —S—, —S(O)—, or —S(O)₂—;
s″ is 0 or 1; and
each of w, x, y, and z are independently 0, 1, 2, 3, or 4.

In some embodiments, an E3 ligase is a DC2 E3 ubiquitin ligase. In some embodiments, the inhibitor compound does not comprise a structure of formula I-c-a-1 or I-c-a-2:

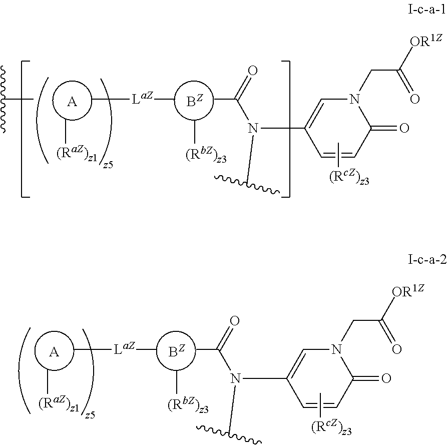

or a pharmaceutically acceptable salt thereof, wherein:
SBM and L are as defined and described above and herein;
R^{1Z} is hydrogen or optionally substituted C₁₋₆ aliphatic;
each R^{aZ}, R^{bZ}, and R^{cZ} are independently hydrogen, R^{AZ}, halogen, —CN, —NO₂, —OR^{Z}—SR^{Z}—NR^{Z}₂, —S(O)₂R^{Z}, —S(O)₂NR^{Z}₂, —S(O)R^{Z}—S(O)(NR^{Z})R^{Z}, —P(O)(OR^{Z})₂, —P(O)(NR^{Z}₂)₂, —CFR^{Z}₂, —CR^{Z}F₂, —CF₃, —CR^{Z}₂(OR^{Z}), —CR^{Z}₂(NR^{Z}₂), —C(O)R^{Z}, —C(O)OR^{Z}, or —C(O)NR^{Z}₂;
each R^{AZ} is independently an optionally substituted group selected from C₁₋₁₀ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{Z} is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R^{Z} groups on the same atom are optionally taken together with their intervening atom to form an optionally substituted 4-11 membered saturated or partially unsaturated carbocyclic or
heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur;
each Ring A^{Z} is independently a bivalent ring selected from phenylenyl, naphthylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
each Ring B^{Z} is independently a bivalent ring selected from phenylenyl, a 3-10 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
L^{aZ} is absent, a covalent bond, or a C₁₋₃ bivalent straight or branched saturated or unsaturated hydrocarbon chain, wherein 1-3 methylene units of the chain are independently and optionally replaced with —O—, —C(O)—, —C(S)—, —C(R^{Z})₂—, —CH(R^{Z})—, —CF(R^{Z})—, —C(F)₂, —N(R^{Z})—, —S—, —S(O)₂— or —CR^{Z}═CR^{Z}—;
z1, z2, and z3 are each independently 0, 1, 2, 3 or 4;
each of z4 and z5 is independently 0 or 1.

In some embodiments, an inhibitor compound binds to and/or inhibits STAT6 protein with measurable affinity. In certain embodiments, an inhibitor compound has an IC₅₀ and/or binding constant of less than about 50 μM, less than about 1 μM, less than about 500 nM, less than about 100 nM, less than about 10 nM, or less than about 1 nM.

### 3. Description of Exemplary Embodiments

In some embodiments, the present invention provides a compound of Formula I-a:

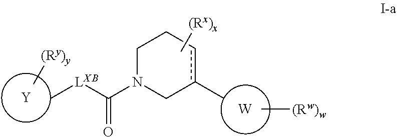

or a pharmaceutically acceptable salt thereof, wherein:
[symbol] is a single or double bond;
Ring W is phenyl, a 5- to 6-membered monocyclic heteroaryl having 1-3 heteroatoms independently selected from nitrogen, oxygen, or sulfur, a 9-membered bicyclic saturated heterocyclyl having 1-3 heteroatoms independently selected from nitrogen, oxygen, or sulfur, 10-membered bicyclic saturated or partially unsaturated heterocyclyl having 1-3 heteroatoms independently selected from nitrogen, oxygen, or sulfur, naphthyl, a 10-membered bicyclic heteroaryl having 1-3 heteroatoms independently selected from nitrogen, oxygen, or sulfur, or a 13-membered tricyclic heteroaryl having 1-3 heteroatoms independently selected from nitrogen, oxygen, or sulfur;
each R^{w} is independently selected from halogen, —CN, —OR^{w1}, —N(R^{w1})₂, —(CH₂)ₙC(O)NR₂, —(CH₂)ₙN(R)C(O)NR₂, —(O)ₘ-phenyl, C₁₋₄ aliphatic, a 6-membered monocyclic saturated or partially unsaturated heterocyclyl having 1-3 heteroatoms independently selected from nitrogen, oxygen, or sulfur, or a 5- to 6-membered monocyclic heteroaryl having 1-3 heteroatoms independently selected from nitrogen, oxygen, or sulfur, wherein each phenyl, heterocyclyl, or heteroaryl is optionally substituted with halogen, C₁₋₄ aliphatic, —OR, or —C(O)NR₂;
each R^{w1} is independently selected from hydrogen, C₁₋₄ aliphatic, phenyl, or 6-membered monocyclic heteroaryl having 1-3 heteroatoms independently selected from nitrogen, oxygen, or sulfur, wherein each aliphatic, phenyl, or heteroaryl is optionally substituted with halogen, —OR, or —C(O)OR;
each R^{x} is independently C₁₋₄ aliphatic, halogen, or —C(O)NR₂;
each R is independently hydrogen or C₁₋₄ aliphatic;
L^{XB} is a bivalent straight or branched C₁₋₃ aliphatic wherein one methylene unit is optionally replaced with —NR—;
Ring Y is a 5-membered monocyclic heteroaryl having 1-3 heteroatoms independently selected from nitrogen, oxygen, or sulfur, phenyl, or a 10-membered bicyclic saturated or partially unsaturated heterocyclyl having 1-3 heteroatoms independently selected from nitrogen, oxygen, or sulfur;
each R^{y} is independently C₁₋₄ aliphatic, halogen, —OR, or —C(O)NR₂;
each m is independently 0 or 1;
each n is independently 0, 1, 2, or 3; and
each w, x, and y is independently 0, 1, 2, 3, or 4,
wherein the compound of formula I-a is not a compound of structure A-B-C defined by the combination of the building blocks A, B, and C within each of Tables (i)-(ii) below:
(i)

| A | B | C |
|---|---|---|
| 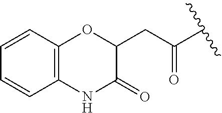 [SMILES: *C(=O)CC1Oc2ccccc2NC1=O] | 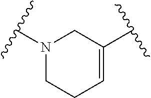 [SMILES: *C1=CCCN(*)C1] | 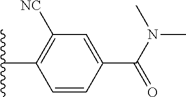 [SMILES: *c1ccc(C(=O)N(C)C)cc1C#N] |
| 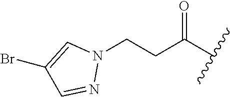 [SMILES: *C(=O)CCn1cc(Br)cn1] | | |

(ii)

| A | B | C |
|---|---|---|
| 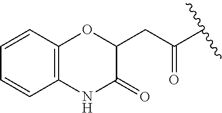 [SMILES: *C(=O)CC1Oc2ccccc2NC1=O] | 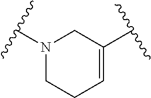 [SMILES: *C1=CCCN(*)C1] | 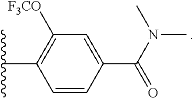 [SMILES: *c1ccc(C(=O)N(C)C)cc1OC(F)(F)F] |

In some embodiments, the present invention provides a compound of formula I-b:

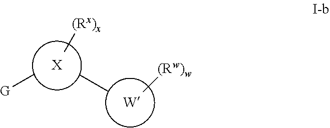

or a pharmaceutically acceptable salt thereof, wherein:
Ring W′ is

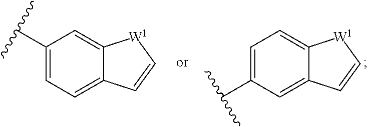

W¹ is O, NH, or NR^{w};
Ring X is a 5-membered monocyclic heteroarylenyl having 1-3 heteroatoms independently selected from nitrogen, oxygen, or sulfur, phenylenyl, or a 5- or 7-membered saturated or partially unsaturated monocyclic heterocyclylenyl having 1-3 heteroatoms independently selected from nitrogen, oxygen, or sulfur;
each R^{w} is independently selected from halogen, —CN, —OR^{w1}, —N(R^{w1})₂, —(CH₂)ₙC(O)NR₂, —(CH₂)ₙN(R)C(O)NR₂, —(O)ₘ-phenyl, C₁₋₄ aliphatic, a 6-membered saturated or partially unsaturated monocyclic heterocyclyl having 1-3 heteroatoms independently selected from nitrogen, oxygen, or sulfur, or a 5- to 6-membered monocyclic heteroaryl having 1-3 heteroatoms independently selected from nitrogen, oxygen, or sulfur, wherein each phenyl, heterocyclyl, or heteroaryl is optionally substituted with halogen, C₁₋₄ aliphatic, —OR, or —C(O)NR₂;
each R^{w1} is independently selected from hydrogen, C₁₋₄ aliphatic, phenyl, or 6-membered monocyclic heteroaryl having 1-3 heteroatoms independently selected from nitrogen, oxygen, or sulfur, wherein each aliphatic, phenyl, or heteroaryl is optionally substituted with halogen, —OR, or —C(O)OR;
each R is independently C₁₋₄ aliphatic, halogen, or —C(O)NR₂;
each R is independently hydrogen or C₁₋₄ aliphatic;
G is hydrogen or

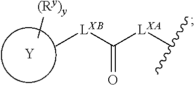

L^{XA} is a covalent bond or a bivalent straight or branched C₁₋₃ aliphatic;
L^{XB} is a bivalent straight or branched C₁₋₃ aliphatic wherein one methylene unit is optionally replaced with —NR—;
Ring Y is a 5-membered monocyclic heteroaryl having 1-3 heteroatoms independently selected from nitrogen, oxygen, or sulfur, phenyl, or a 10-membered bicyclic saturated or partially unsaturated heterocyclyl having 1-3 heteroatoms independently selected from nitrogen, oxygen, or sulfur;
each R^{y} is independently C₁₋₄ aliphatic, halogen, —OR, or —C(O)NR₂;
each m is independently 0 or 1;
each n is independently 0, 1, 2, or 3; and
each w, x, and y is independently 0, 1, 2, 3, or 4.

In some embodiments, the inhibitor compound of formula I-a or I-b specifically affects, as its primary mechanism of action, the inhibition of STAT6.

As described above and defined herein, Ring W is phenyl, a 5- to 6-membered monocyclic heteroaryl having 1-3 heteroatoms independently selected from nitrogen, oxygen, or sulfur, a 9-membered bicyclic saturated heterocyclyl having 1-3 heteroatoms independently selected from nitrogen, oxygen, or sulfur, 10-membered bicyclic saturated or partially unsaturated heterocyclyl having 1-3 heteroatoms independently selected from nitrogen, oxygen, or sulfur, naphthyl, a 10-membered bicyclic heteroaryl having 1-3 heteroatoms independently selected from nitrogen, oxygen, or sulfur, or a 13-membered tricyclic heteroaryl having 1-3 heteroatoms independently selected from nitrogen, oxygen, or sulfur.

In some embodiments, Ring W is phenyl or naphthyl. In some embodiments, Ring W is phenyl. In some embodiments, Ring W is naphthyl.

In some embodiments, Ring W is a 9-membered bicyclic saturated heterocyclyl with 1-3 heteroatoms independently selected from nitrogen, oxygen, and sulfur. In some embodiments, Ring W is a 9-membered bicyclic saturated heterocyclyl with 1-2 heteroatoms independently selected from nitrogen, oxygen, and sulfur.

In some embodiments, Ring W is a 10-membered saturated or partially unsaturated bicyclic heterocyclyl with 1-3 heteroatoms independently selected from nitrogen, oxygen, and sulfur. In some embodiments, Ring W is a 10-membered saturated or partially unsaturated bicyclic heterocyclyl with 1-2 heteroatoms independently selected from nitrogen, oxygen, and sulfur. In some embodiments, Ring W is chromanyl, chromenyl, isochromenyl, tetrahydroisoquinolinyl, tetrahydroquinolinylyl, 1,2-dihydroquinolinyl, or 1,2-dihydroisoquinolinyl.

In some embodiments, Ring W is a 5-6 membered heteroaryl with 1-3 heteroatoms independently selected from nitrogen, oxygen, and sulfur. In some embodiments, Ring W is a 5-membered heteroaryl with 1-3 heteroatoms independently selected from nitrogen, oxygen, and sulfur. In some embodiments, Ring W is furanyl, oxazolyl, isoxazolyl, or oxadiazolyl. In some embodiments, Ring W is a 6-membered heteroaryl with 1-3 heteroatoms independently selected from nitrogen, oxygen, and sulfur. In some embodiments, Ring W is pyridinyl, pyrimidinonyl, pyridazinyl, or triazinyl.

In some embodiments, Ring W is a 10-membered bicyclic heteroaryl with 1-3 heteroatoms independently selected from nitrogen, oxygen, and sulfur. In some embodiments, Ring W is a 10-membered bicyclic heteroaryl with 1-2 heteroatoms independently selected from nitrogen, oxygen, and sulfur. In some embodiments, Ring W is a 10-membered bicyclic heteroaryl with 1-2 nitrogen heteroatoms. In some embodiments, Ring W is quinolinyl, isoquinolinyl, quinolizinyl, quinoxalinyl, phthalazinyl, quinazolinyl, cinnolinyl, or 1,8-naphthyridinyl.

In some embodiments, Ring W is a 13-membered tricyclic heteroaryl with 1-3 heteroatoms independently selected form nitrogen, oxygen, and sulfur. In some embodiments, Ring W is 3H-benzo[e]indolyl.

In some embodiments, Ring W is

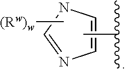

In some embodiments, Ring W is

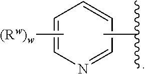

In some embodiments, Ring W is

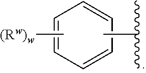

In some embodiments, Ring W is

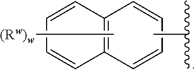

In some embodiments, Ring W is

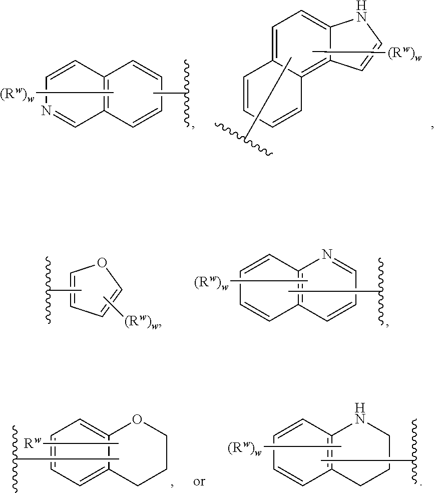

In some embodiments, Ring W is

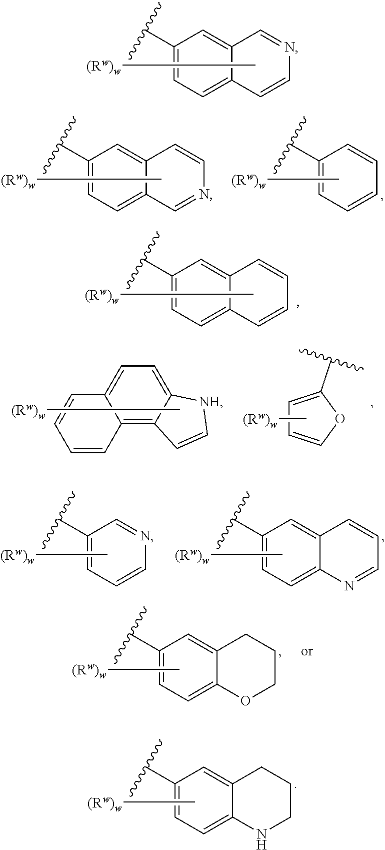

In some embodiments, Ring W is

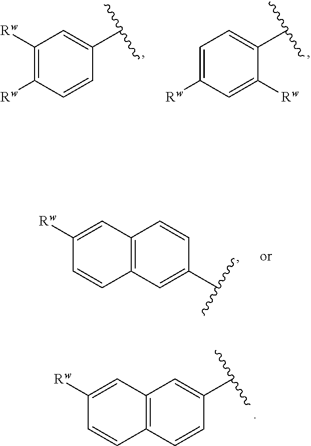

In some embodiments, Ring W is

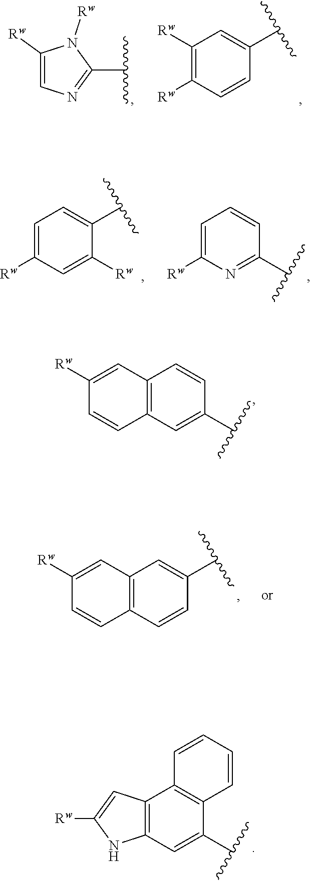

In some embodiments, Ring W is

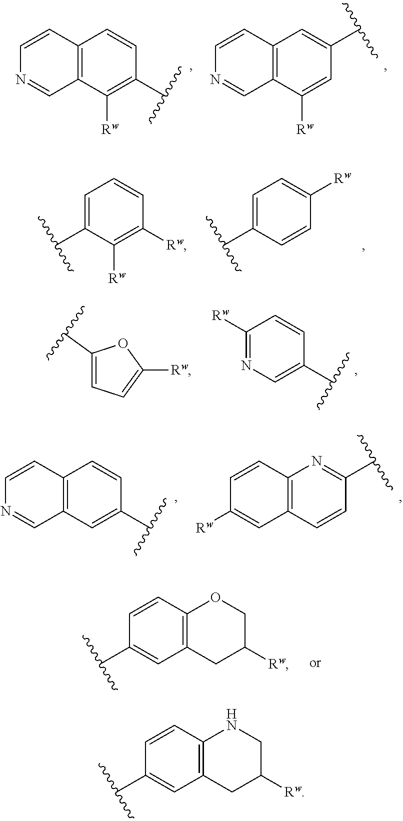

In some embodiments, Ring W is

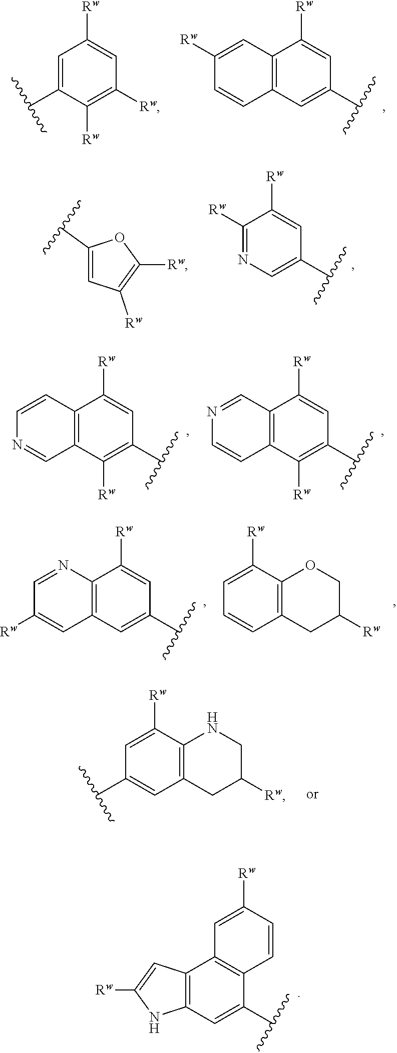

In some embodiments, Ring W is as depicted in the compounds of Table 1A or Table 1B, below.

In some embodiments, Ring W and its R^{w} substituents are

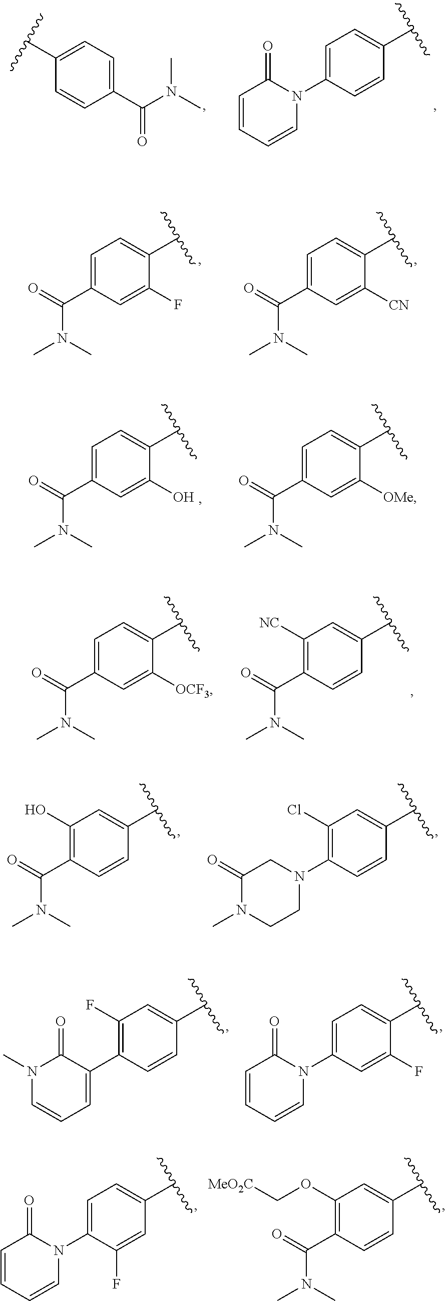

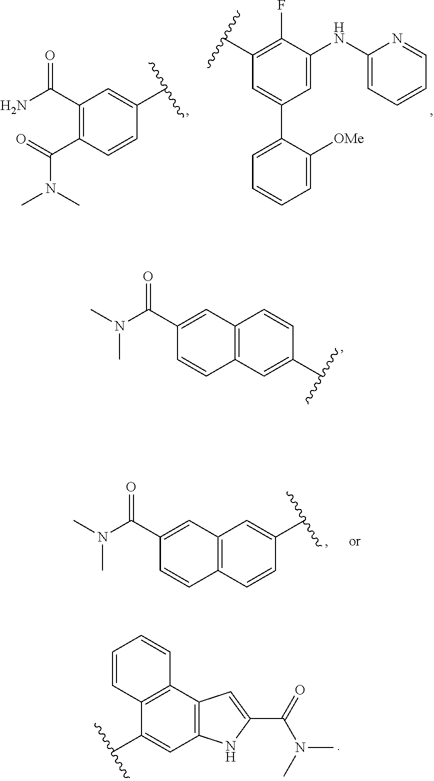 [SMILES: *c1ccc(C(=O)N(C)C)c(C(N)=O)c1 ; *c1cc(-c2ccccc2OC)cc(Nc2ccccn2)c1F ; *c1ccc2cc(C(=O)N(C)C)ccc2c1 ; *c1ccc2ccc(C(=O)N(C)C)cc2c1 ; *c1cc2[nH]c(C(=O)N(C)C)cc2c2ccccc12]

In some embodiments, Ring W and its R^{w} substituents are

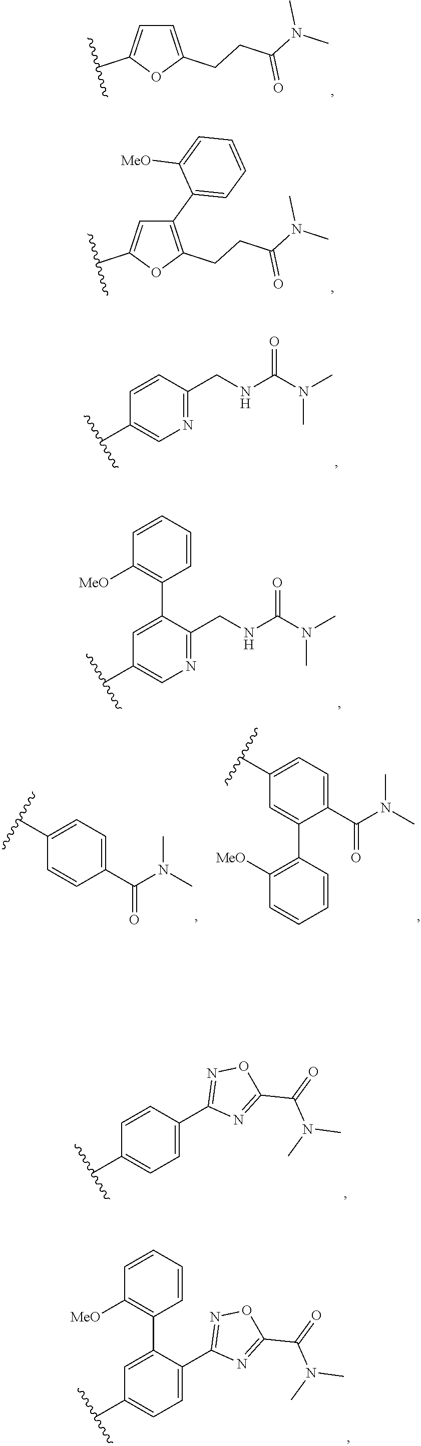

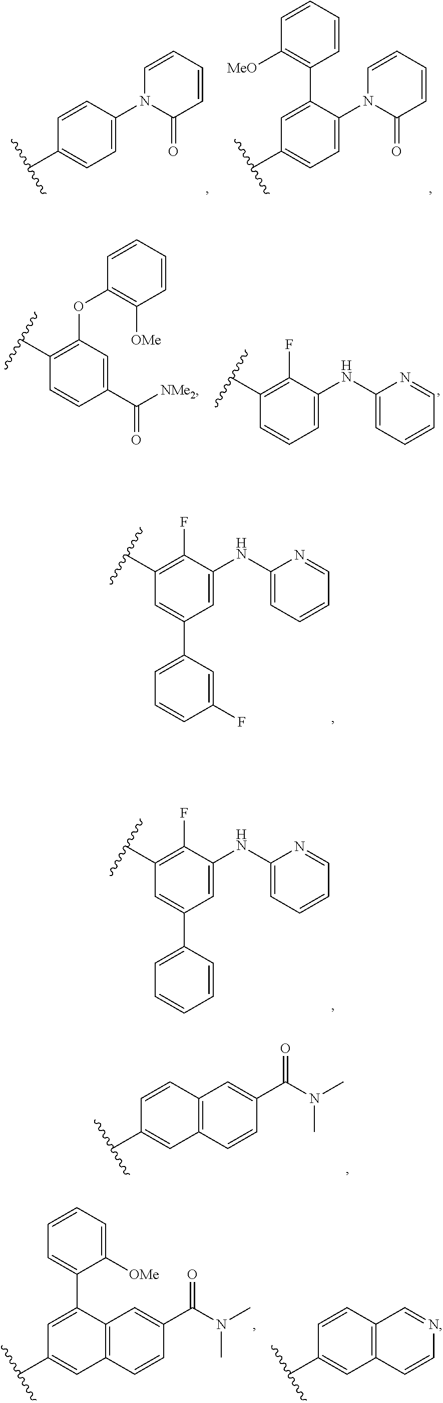

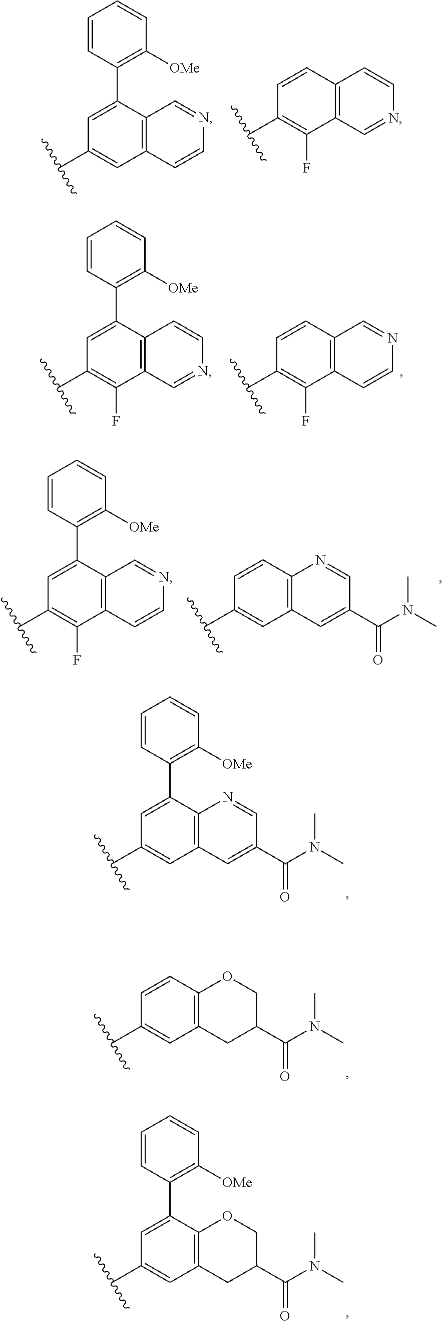

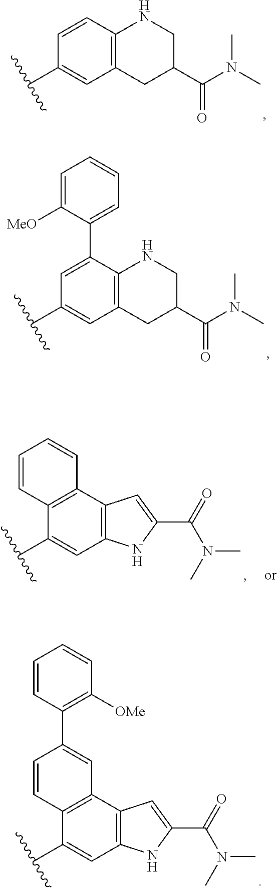 [SMILES: *c1ccc2c(c1)CC(C(=O)N(C)C)CN2 ; *c1cc2c(c(-c3ccccc3OC)c1)NCC(C(=O)N(C)C)C2 ; *c1cc2[nH]c(C(=O)N(C)C)cc2c2ccccc12 ; *c1cc2[nH]c(C(=O)N(C)C)cc2c2cc(-c3ccccc3OC)ccc12]

As described above and defined herein, W¹ is O, NH, or NR^{w}. In some embodiments, W¹ is O. In some embodiments, W¹ is NH or NR^{w}. In some embodiments, W¹ is NH. In some embodiments, W¹ is NR^{w}.

As described above and defined herein, Ring W′ is

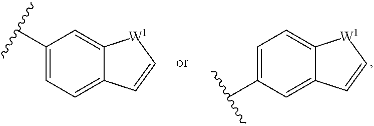

wherein W¹ is as defined herein.

In some embodiments, Ring W′ is

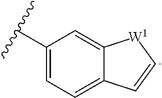

In some embodiments, Ring W′ is

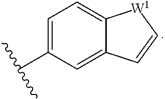

In some embodiments, Ring W′ and its R^{w} substituents is

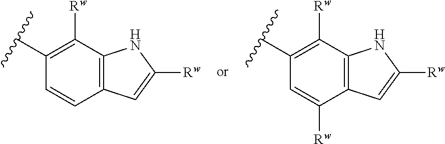

In some embodiments, Ring W′ and its R^{w} substituents is

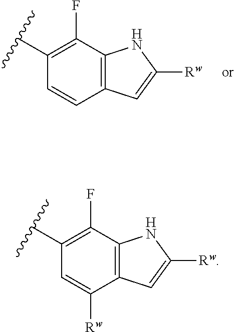

In some embodiments, Ring W′ and its R^{w} substituents is

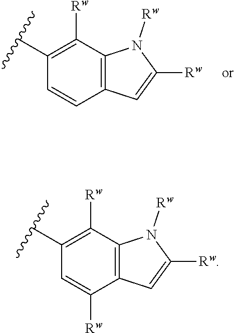

In some embodiments, Ring W′ and its R^{w} substituents is

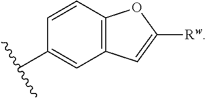

In some embodiments, Ring W′ and its R^{w} substituents is

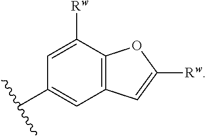

In some embodiments, Ring W′ and its R^{w} substituents is

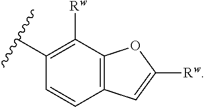

In some embodiments, Ring W′ and its R^{w} substituents is

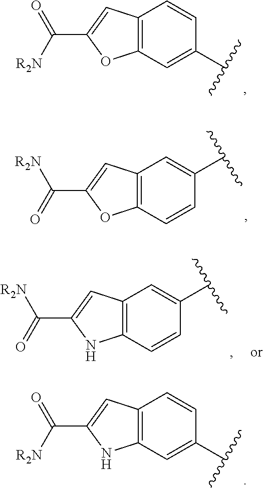

In some embodiments, Ring W′ and its R^{w} substituents is

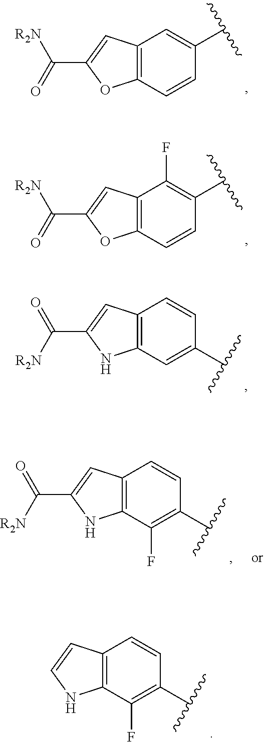

In some embodiments, Ring W′ is as depicted in the compounds of Table 1A or Table 1B, below.

In some embodiments, Ring W′ is

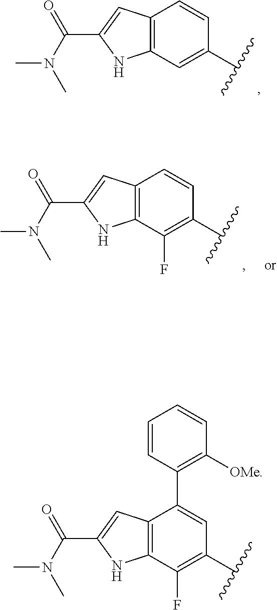 [SMILES: *c1ccc2cc(C(=O)N(C)C)[nH]c2c1 ; *c1ccc2cc(C(=O)N(C)C)[nH]c2c1F ; *c1cc(-c2ccccc2OC)c2cc(C(=O)N(C)C)[nH]c2c1F]

As described above and defined herein, [symbol] is a single or double bond. In some embodiments, [symbol] is a single bond. In some embodiments, [symbol] is a double bond.

As described above and defined herein, compounds of formula I-a comprise a moiety:

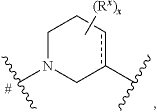

wherein # represents the point of attachment to G, and wherein R^{x} and x are as defined herein.

In some embodiments, a moiety

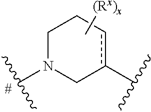

is

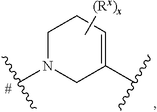

wherein # represents the point of attachment to G. In some embodiments, a moiety

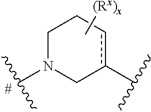

is

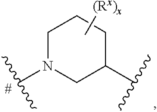

wherein # represents the point of attachment to G. In some embodiments, a moiety

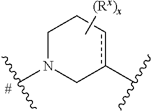

is

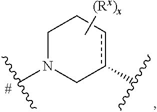

wherein # represents the point of attachment to G. In some embodiments, a moiety

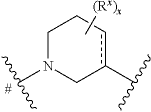

is

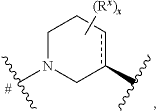

wherein # represents the point of attachment to G.

In some embodiments, a moiety

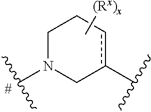

is as depicted in the compounds of Table 1A or Table 1B, below.

In some embodiments, a moiety

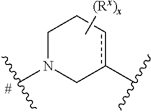

is

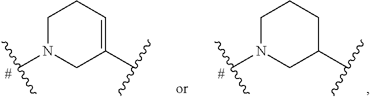 [SMILES: *C1=CCCN(*)C1 ; *C1CCCN(*)C1]

wherein # represents the point of attachment to G. In some embodiments, a moiety

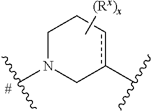

is

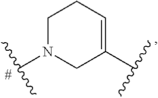 [SMILES: *C1=CCCN(*)C1]

wherein # represents the point of attachment to G. In some embodiments, a moiety

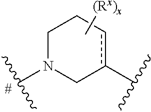

is

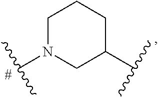 [SMILES: *C1CCCN(*)C1]

wherein # represents the point of attachment to G.

As described above and defined herein, Ring X is a 5-membered monocyclic heteroarylenyl having 1-3 heteroatoms independently selected from nitrogen, oxygen, or sulfur, phenylenyl, or a 5- or 7-membered saturated or partially unsaturated monocyclic heterocyclylenyl having 1-3 heteroatoms independently selected from nitrogen, oxygen, or sulfur.

In some embodiments, Ring X is a 5-membered heteroarylenyl having 1-3 heteroatoms independently selected from nitrogen, oxygen, or sulfur. In some embodiments, Ring X is a thiazole ring.

In some embodiments, Ring X is phenylenyl.

In some embodiments, Ring X is a 5- or 7-membered saturated or partially unsaturated monocyclic heterocyclylenyl having 1-3 heteroatoms independently selected from nitrogen, oxygen, or sulfur. In some embodiments, Ring X is a 5-membered saturated or partially unsaturated monocyclic heterocyclylenyl having 1-3 heteroatoms independently selected from nitrogen, oxygen, or sulfur. In some embodiments, Ring X is a 5-membered saturated or partially unsaturated monocyclic heterocyclylenyl having 1 heteroatom selected from nitrogen, oxygen, or sulfur. In some embodiments, Ring X is a pyrrolidine ring. In some embodiments, Ring X is a 7-membered saturated or partially unsaturated monocyclic heterocyclylenyl having 1-3 heteroatoms independently selected from nitrogen, oxygen, or sulfur. In some embodiments, Ring X is a 7-membered saturated or partially unsaturated monocyclic heterocyclylenyl having 1 heteroatom selected from nitrogen, oxygen, or sulfur. In some embodiments, Ring X is an azepane ring.

In some embodiments, Ring X and its R^{x} substituents are

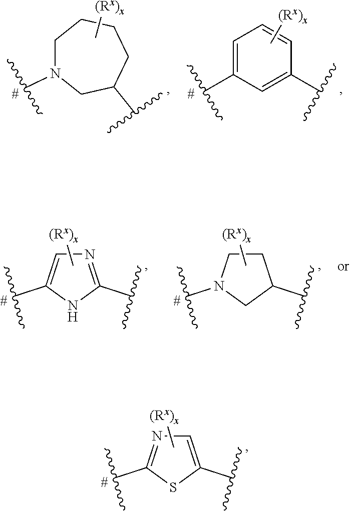

wherein # represents the point of attachment to G.

In some embodiments, Ring X and its R^{x} substituents are

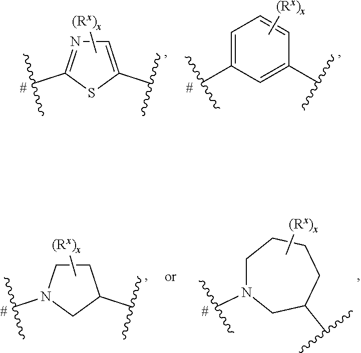

wherein # represents the point of attachment to G.

In some embodiments, Ring X is as depicted in the compounds of Table 1A or Table 1B, below.

In some embodiments, Ring X and its R^{X} substituents are

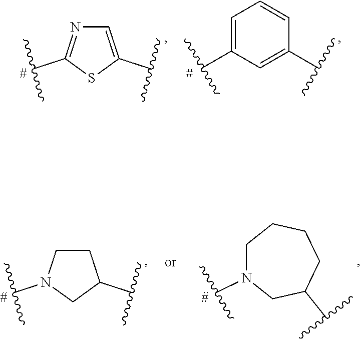 [SMILES: *c1cnc(*)s1 ; *c1cccc(*)c1 ; *C1CCN(*)C1 ; *C1CCCCN(*)C1]

wherein # represents the point of attachment to G.

As described above and defined herein, G is hydrogen or

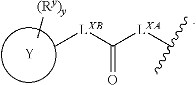

In some embodiments, G is hydrogen. In some embodiments, G is

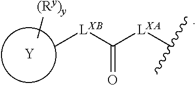

In some embodiments, G is as depicted in the compounds of Table 1A or Table 1B, below.

As described above and defined herein, L^{XA} is a covalent bond or a bivalent straight or branched C₁₋₃ aliphatic.

In some embodiments, L^{XA} is a covalent bond. In some embodiments, L^{XA} is a bivalent straight or branched C₁₋₃ aliphatic. In some embodiments, L^{XA} is straight or branched C₁₋₃ alkylene. In some embodiments, L^{XA} is straight or branched C₁₋₂ alkylene. In some embodiments, L^{XA} is —CH₂—.

In some embodiments, L^{XA} is as depicted in the compounds of Table 1A or Table 1B, below.

As described above and defined herein, L^{XB} is a bivalent straight or branched C₁₋₃ aliphatic wherein one methylene unit is optionally replaced with —NR—.

In some embodiments, L^{XB} is straight or branched C₁₋₃ alkylene wherein one methylene unit is optionally replaced with —NR—. In some embodiments, L^{XB} is straight or branched C₁₋₂ alkylene wherein one methylene unit is optionally replaced with —NR—. In some embodiments, L^{XB} is a bivalent straight or branched C₁₋₃ aliphatic. In some embodiments, L^{XB} is straight or branched C₁₋₃ alkylene. In some embodiments, L^{XB} is straight or branched C₁₋₂ alkylene. In some embodiments, L^{XB} is —CH₂—. In some embodiments, L^{XB} is —CH₂CH₂—. In some embodiments, L^{XB} is a bivalent straight or branched C₁₋₃ aliphatic wherein one methylene unit is replaced with —NR—. In some embodiments, L^{XB} is —NH—.

In some embodiments, L^{XB} is as depicted in the compounds of Table 1A or Table 1B, below.

As described above and defined herein, Ring Y is a 5-membered monocyclic heteroaryl having 1-3 heteroatoms independently selected from nitrogen, oxygen, or sulfur, phenyl, or a 10-membered bicyclic saturated or partially unsaturated heterocyclyl having 1-3 heteroatoms independently selected from nitrogen, oxygen, or sulfur.

In some embodiments, Ring Y is a 5-membered monocyclic heteroaryl having 1-3 heteroatoms independently selected from nitrogen, oxygen, or sulfur. In some embodiments, Ring Y is a 5-membered monocyclic heteroaryl ring with 1 heteroatom selected from nitrogen, oxygen, and sulfur. In some embodiments, Ring Y is a 5-membered monocyclic heteroaryl ring with 2 heteroatoms independently selected from nitrogen, oxygen, and sulfur. In some embodiments, Ring Y is a 5-membered monocyclic heteroaryl ring with 3 heteroatoms independently selected from nitrogen, oxygen, and sulfur. In some embodiments, Ring Y is a 5-membered monocyclic heteroaryl ring with 2-3 nitrogen heteroatoms. In some embodiments, Ring Y is a triazolyl.

In some embodiments, Ring Y is phenyl.

In some embodiments, Ring Y is a 10-membered bicyclic saturated or partially unsaturated heterocyclyl having 1-3 heteroatoms independently selected from nitrogen, oxygen, or sulfur. In some embodiments, Ring Y is 3,4-dihydro-2H-benzo[b][1,4]oxazinyl.

In some embodiments Ring Y is

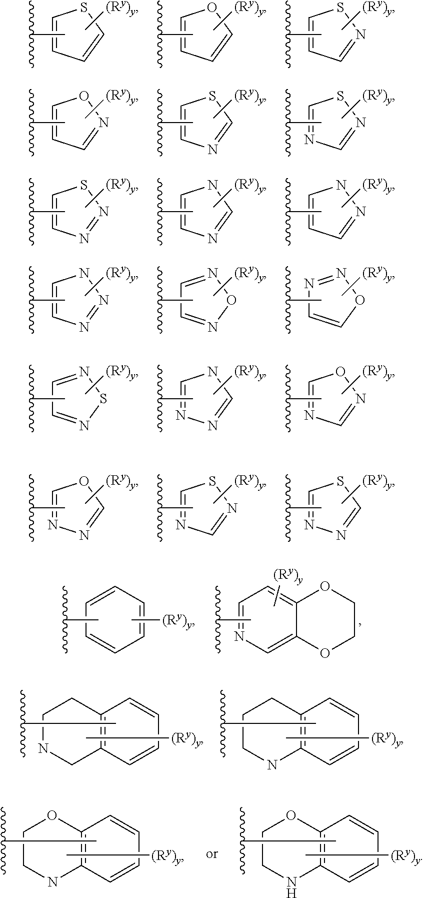

In some embodiments, Ring Y is

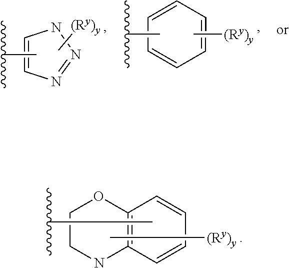

In some embodiments, Ring Y is

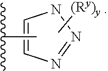

In some embodiments, Ring Y is

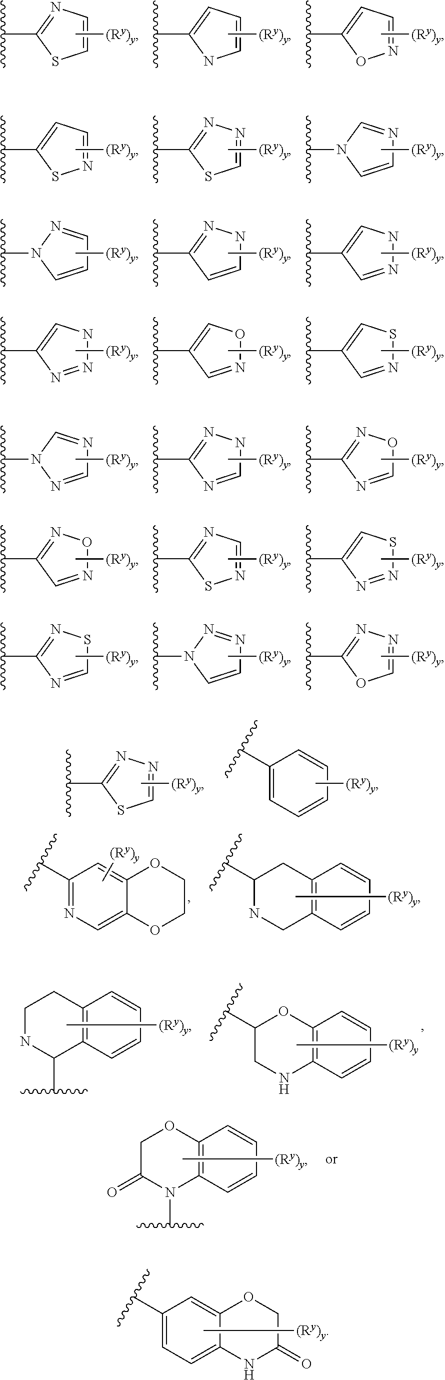

In some embodiments, Ring Y is

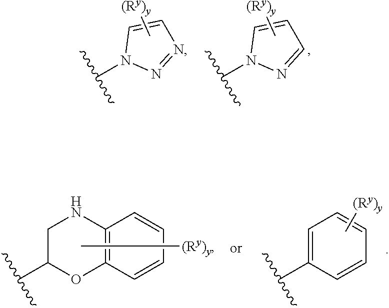

In some embodiments, Ring Y is

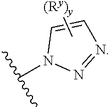

In some embodiment, Ring Y is as depicted in the compounds of Table 1A or Table 1B, below.

In some embodiments, Ring Y and its R^{y} substituents are

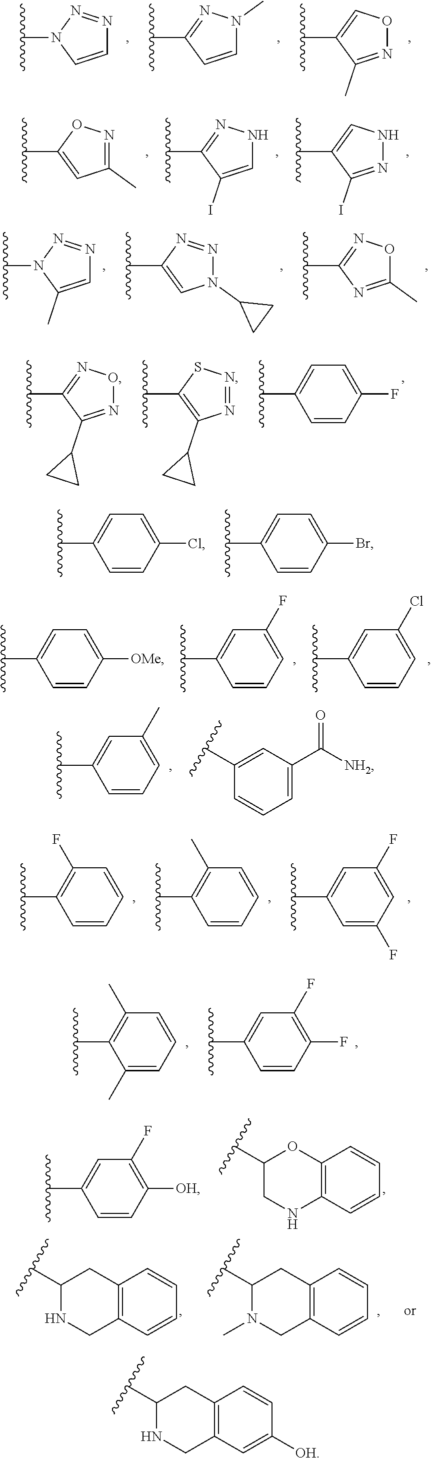

In some embodiments, Ring Y is

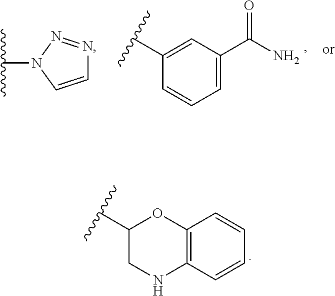

In some embodiments, Ring Y is

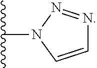 [SMILES: *n1ccnn1]

As described above and defined herein, each R^{w} is independently selected from halogen, —CN, —OR^{w1}, —N(R^{w1})₂, —(CH₂)ₙC(O)NR₂, —(CH₂)ₙN(R)C(O)NR₂, —(O)ₘ-phenyl, C₁₋₄ aliphatic, a 6-membered monocyclic saturated or partially unsaturated heterocyclyl having 1-3 heteroatoms independently selected from nitrogen, oxygen, or sulfur, or a 5- to 6-membered monocyclic heteroaryl having 1-3 heteroatoms independently selected from nitrogen, oxygen, or sulfur, wherein each phenyl, heterocyclyl, or heteroaryl is optionally substituted with halogen, C₁₋₄ aliphatic, —OR, or —C(O)NR₂.

In some embodiments, a R^{w} is halogen. In some embodiments, a R^{w} is fluoro, chloro, or bromo. In some embodiments, a R^{w} is fluoro.

In some embodiments, a R^{w} is —CN.

In some embodiments, a R^{w} is —OR^{w1}. In some embodiments, a R^{w} is —OR^{w1}, wherein R^{w1} of R^{w} is hydrogen, C₁₋₄ aliphatic optionally substituted with one or more halogen or —C(O)OR, or phenyl optionally substituted with one or more —OR. In some embodiments, a R^{w} is —OR^{w1}, wherein R^{w1} of R^{w} is hydrogen or C₁₋₄ aliphatic.

In some embodiments, a R^{w} is —N(R^{w1})₂. In some embodiments, a R^{w} is —N(H)(R^{w1}). In some embodiments, a R^{w} is —N(R^{w1})₂, wherein each R^{w1} of R^{w} is independently hydrogen, C₁₋₄ aliphatic, or 6-membered monocyclic heteroaryl having 1-3 heteroatoms independently selected from nitrogen, oxygen, or sulfur. In some embodiments, a R^{w} is —N(R^{w1})₂, wherein each R^{w1} of R^{w} is independently hydrogen or 6-membered monocyclic heteroaryl having 1-3 heteroatoms independently selected from nitrogen, oxygen, or sulfur (e.g., a pyridyl).

In some embodiments, a R^{w} is —(CH₂)ₙC(O)NR₂. In some embodiments, a R^{w} is —(CH₂)ₙC(O)N(H)R. In some embodiments, a R^{w} is —C(O)NR₂. In some embodiments, a R^{w} is —(CH₂)₂C(O)NR₂. In some embodiments, a R^{w} is —C(O)NH₂. In some embodiments, a R^{w} is —C(O)N(CH₃)₂.

In some embodiments, a R^{w} is —(CH₂)ₙC(O)N(CH₃)₂.

In some embodiments, a R^{w} is —(CH₂)ₙN(R)C(O)NR₂. In some embodiments, a R^{w} is —N(R)C(O)NR₂. In some embodiments, a R^{w} is —CH₂N(R)C(O)NR₂. In some embodiments, a R^{w} is —(CH₂)ₙN(H)C(O)N(CH₃)₂.

In some embodiments, a R^{w} is —(O)ₘ-phenyl, wherein the phenyl is optionally substituted with halogen, C₁₋₄ aliphatic, —OR, or —C(O)NR₂. In some embodiments, a R^{w} is —(O)ₘ-phenyl, wherein the phenyl is optionally substituted with halogen, —OR, or —C(O)NR₂. In some embodiments, a R^{w} is phenyl, wherein the phenyl is optionally substituted with halogen, C₁₋₄ aliphatic, —OR, or —C(O)NR₂. In some embodiments, a R^{w} is phenyl, wherein the phenyl is optionally substituted with halogen, —OR, or —C(O)NR₂. In some embodiments, a R^{w} is —O-phenyl, wherein the phenyl is optionally substituted with halogen, C₁₋₄ aliphatic, —OR, or —C(O)NR₂. In some embodiments, a R^{w} is phenyl, wherein the phenyl is optionally substituted with —OR (e.g., —OCH₃).

In some embodiments, a R^{w} is C₁₋₄ aliphatic. In some embodiments, a R^{w} is C₁₋₄ alkyl.

In some embodiments, a R^{w} is a 6-membered monocyclic saturated or partially unsaturated heterocyclyl having 1-3 heteroatoms independently selected from nitrogen, oxygen, or sulfur, wherein the heterocyclyl is optionally substituted with halogen, C₁₋₄ aliphatic, —OR, or —C(O)NR₂. In some embodiments, a R^{w} is a 6-membered monocyclic saturated or partially unsaturated heterocyclyl having 1-2 heteroatoms independently selected from nitrogen, oxygen, or sulfur, wherein the heterocyclyl is optionally substituted with halogen, C₁₋₄ aliphatic, —OR, or —C(O)NR₂. In some embodiments, a R^{w} is a 6-membered monocyclic saturated or partially unsaturated heterocyclyl having 1-3 heteroatoms independently selected from nitrogen, oxygen, or sulfur, wherein the heterocyclyl is optionally substituted with C₁₋₄ aliphatic. In some embodiments R^{w} is a piperidinyl, piperazinyl, tetrahydropyranyl, 2H-pyranyl, 4H-pyranyl, 1,4-dioxanyl, 1,4-dioxinyl, thianyl, 2H-thiopyranyl, 4H-thiopyranyl, 1,3-dithanyl, 1,4-dithanyl, morpholinyl, thiomorpholinyl, dihydropyridinyl, tetrahydropyridinyl, dihydropyranyl, dihydrothiopyranyl, or tetrahydrothiopyranyl, each of which is optionally substituted with halogen, C₁₋₄ aliphatic, —OR, or —C(O)NR₂. In some embodiments, a R^{w} is a piperazinyl optionally substituted with C₁₋₄ aliphatic (e.g., C₁₋₄ alkyl).

In some embodiments, a R^{w} is a 5- to 6-membered monocyclic heteroaryl having 1-3 heteroatoms independently selected from nitrogen, oxygen, or sulfur, wherein the heteroaryl is optionally substituted with halogen, C₁₋₄ aliphatic, —OR, or —C(O)NR₂. In some embodiments, a R^{w} is a 5-membered monocyclic heteroaryl having 1-3 heteroatoms independently selected from nitrogen, oxygen, or sulfur, wherein the heteroaryl is optionally substituted with halogen, C₁₋₄ aliphatic, —OR, or —C(O)NR₂. In some embodiments, a R^{w} is a 5-membered monocyclic heteroaryl having 1-3 heteroatoms independently selected from nitrogen, oxygen, or sulfur, wherein the heteroaryl is optionally substituted with —C(O)NR₂. In some embodiments, R^{w} is a pyrazolyl, imidazolyl, triazolyl, tetrazolyl, furanyl, thiophenyl, oxazolyl, isoxazolyl, isothiazolyl, thiazolyl, oxadiazolyl, or thiadiazolyl, each of which is optionally substituted with halogen, C₁₋₄ aliphatic, —OR, or —C(O)NR₂. In some embodiments, a R^{w} is an oxadiazolyl optionally substituted with —C(O)NR₂ (e.g., —C(O)N(CH₃)₂. In some embodiments, a R^{w} is a 6-membered monocyclic heteroaryl having 1-3 heteroatoms independently selected from nitrogen, oxygen, or sulfur, wherein the heteroaryl is optionally substituted with halogen, C₁₋₄ aliphatic, —OR, or —C(O)NR₂. In some embodiments, a R^{w} is a 6-membered monocyclic heteroaryl having 1-3 heteroatoms independently selected from nitrogen, oxygen, or sulfur, wherein the heteroaryl is optionally substituted with C₁₋₄ aliphatic. In some embodiments, R^{w} is optionally substituted pyridinyl, pyrimidinyl, pyridazinyl, triazinyl, pyridinonyl, pyrazinonyl, or pyrimidinoyl. In some embodiments, a R^{w} is a pyridonyl optionally substituted with C₁₋₄ aliphatic.

In some embodiments, R^{w} is as depicted in the compounds of Table 1A or Table 1B, below.

In some embodiments, R^{w} is fluoro, chloro, —CN, —OH, —OMe, —OCH₂CO₂Me, —C(O)NH₂, —C(O)NHMe, —C(O)NHEt, —C(O)NHnPr, —C(O)NMe₂, —C(O)N(Me)Et, —C(O)N(Me)nPr,

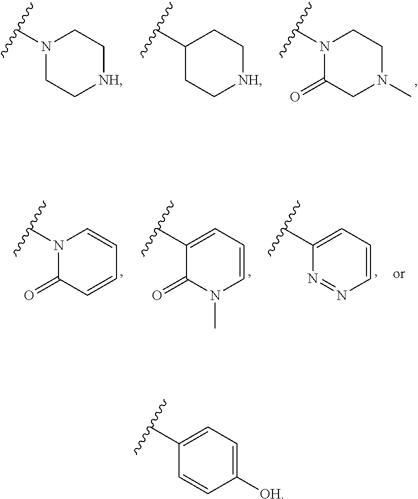

In some embodiments, R^{w} is fluoro, chloro, —CN, methyl, —CF₃, —CHF₂, —OH, —OMe, —OCH₂CO₂Me, —C(O)NH₂, —C(O)NHMe, —C(O)NHEt, —C(O)NHnPr, —C(O)NMe₂, —C(O)N(Me)Et, —C(O)N(Me)nPr,

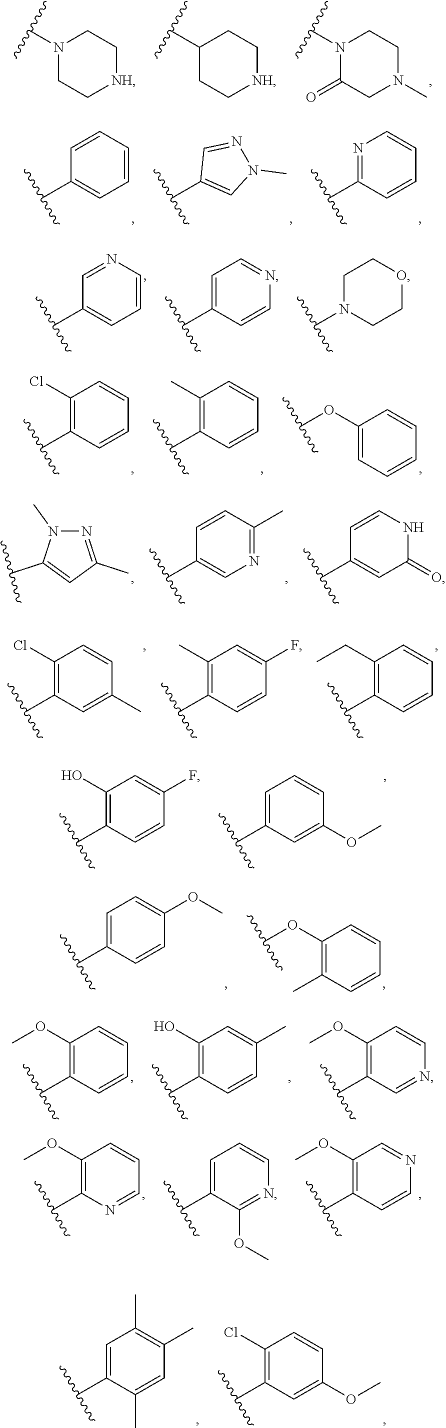

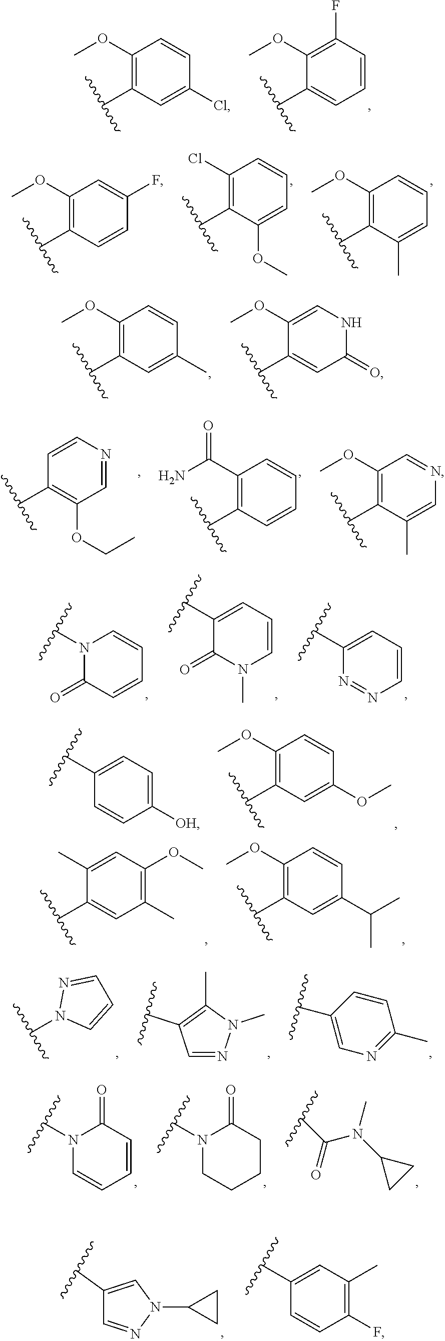

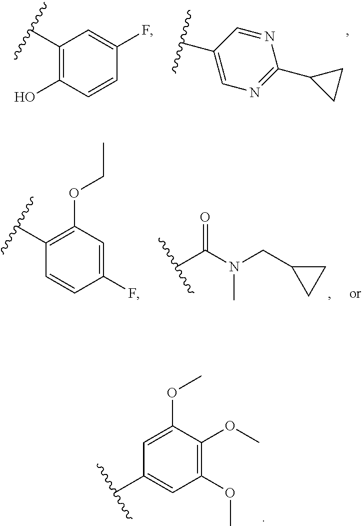

In some embodiments, R^{w} is

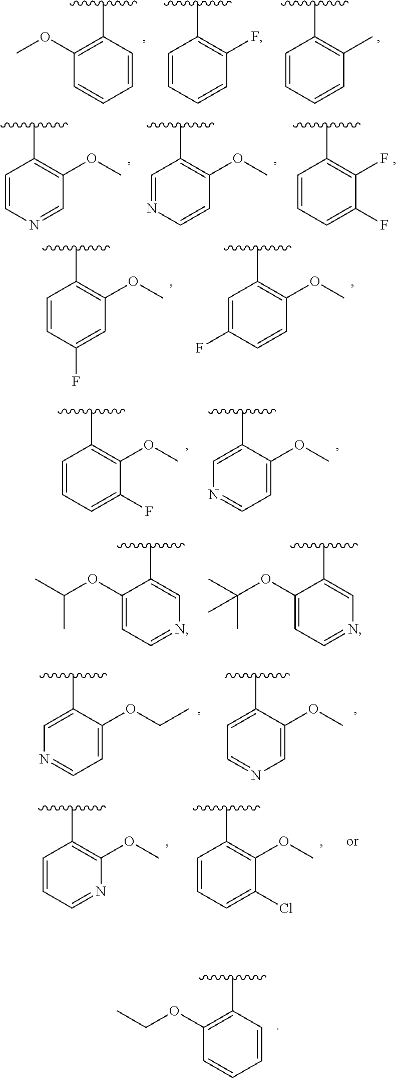

In some embodiments, R^{w} is

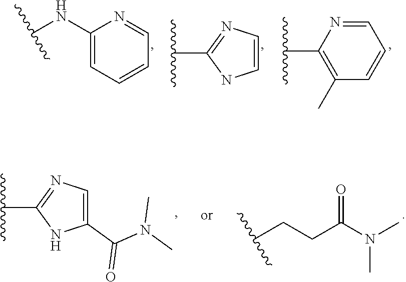

In some embodiments, R^{w} is —CH₂CH₂CH₃,

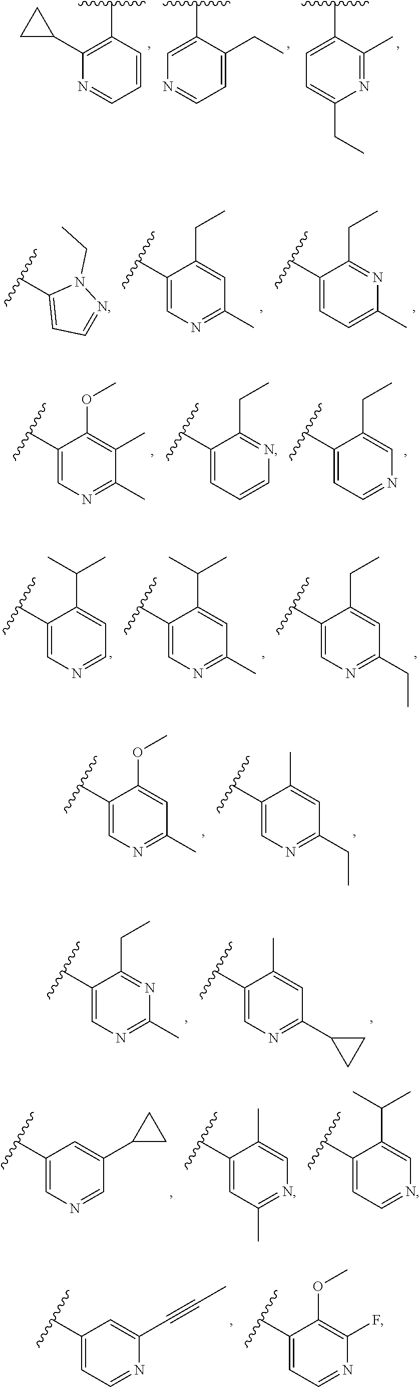

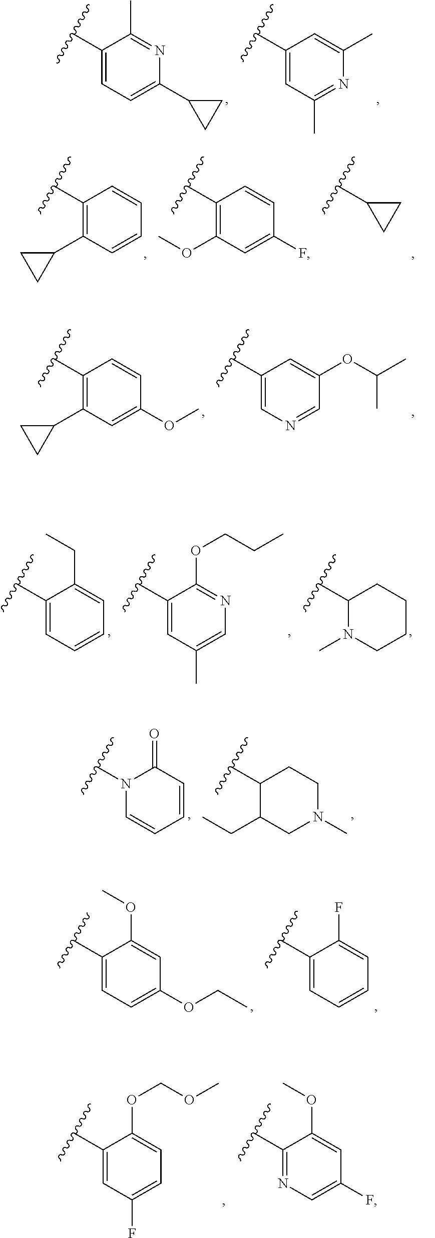

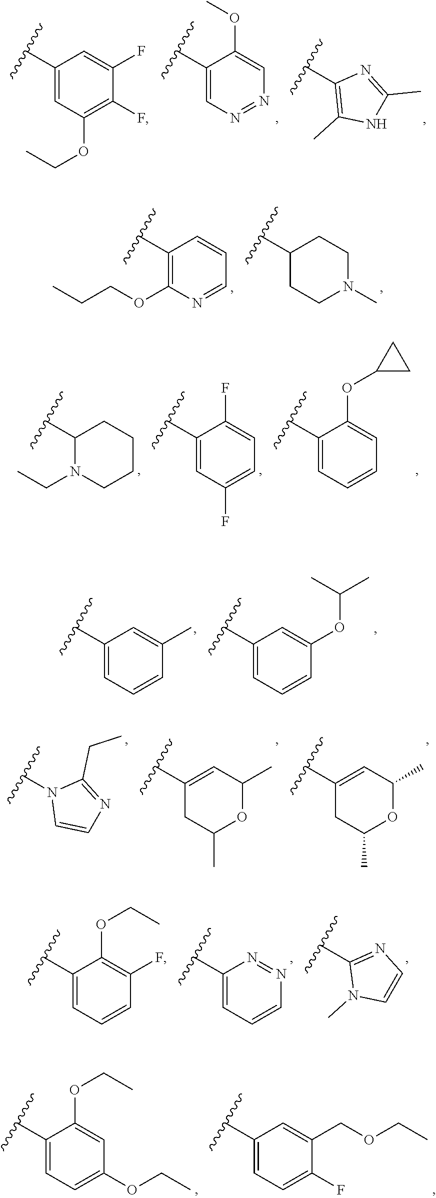

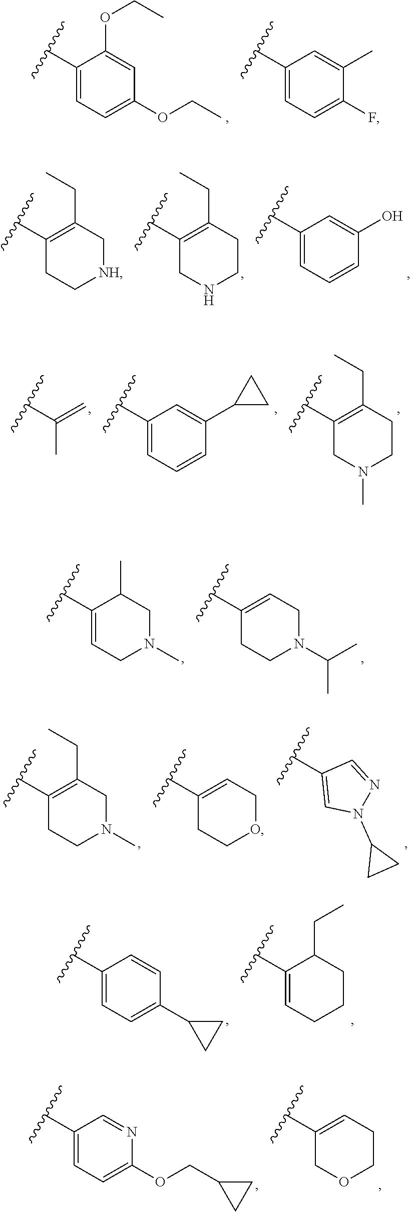

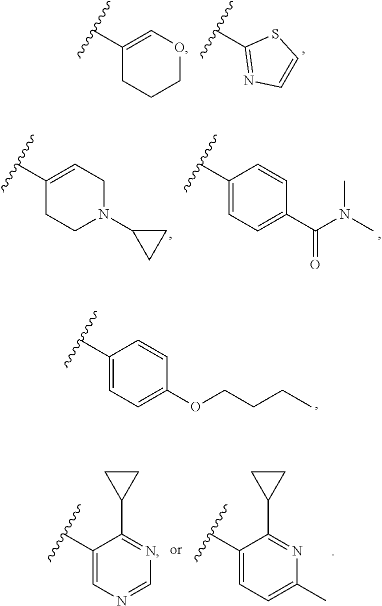

In some embodiments, R^{w} is

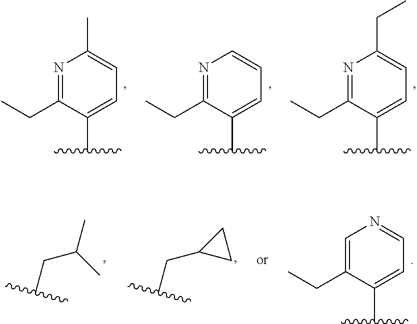

In some embodiments, R^{w} is fluoro, chloro, —CN, —OH, —OMe, —OCF₃, —OCH₂CO₂Me, —C(O)NH₂, —C(O)NMe₂, —(CH₂)₂C(O)NMe₂, —CH₂N(H)C(O)NMe₂,

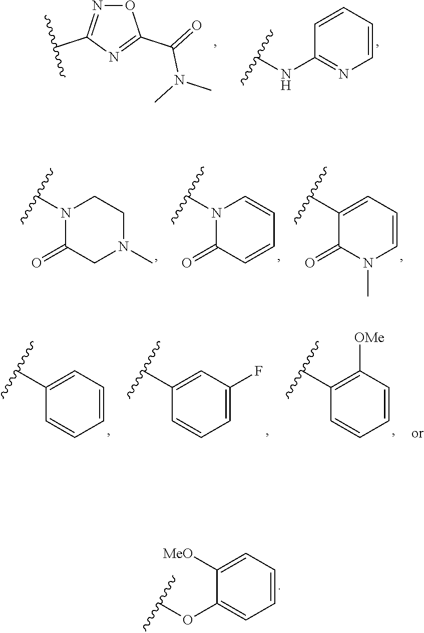

As described above and defined herein, each R^{w1} is independently selected from hydrogen, C₁₋₄ aliphatic, phenyl, or 6-membered monocyclic heteroaryl having 1-3 heteroatoms independently selected from nitrogen, oxygen, or sulfur, wherein each aliphatic, phenyl, or heteroaryl is optionally substituted with halogen, —OR, or —C(O)OR.

In some embodiments, each R^{w1} is independently selected from hydrogen or C₁₋₄ aliphatic.

In some embodiments, a R^{w1} is hydrogen.

In some embodiments, a R^{w1} is C₁₋₄ aliphatic, wherein the aliphatic is optionally substituted with halogen, —OR, or —C(O)OR. In some embodiments, a R^{w1} is C₁₋₄ alkyl, wherein the alkyl is optionally substituted with halogen, —OR, or —C(O)OR. In some embodiments, a R^{w1} is C₁₋₄ aliphatic (e.g., —CH₃). In some embodiments, a R^{w1} is C₁₋₄ aliphatic, wherein the aliphatic is optionally substituted with halogen (e.g., —CF₃). In some embodiments, a R^{w1} is C₁₋₄ aliphatic, wherein the aliphatic is optionally substituted with —C(O)OR (e.g., —CH₂C(O)OCH₃).

In some embodiments, a R^{w1} is phenyl, wherein the phenyl is optionally substituted with halogen, —OR, or —C(O)OR. In some embodiments, a R^{w1} is phenyl. In some embodiments, a R^{w1} is phenyl, wherein the phenyl is optionally substituted with —OR (e.g., —OCH₃).

In some embodiments, a R^{w1} is a 6-membered monocyclic heteroaryl having 1-3 heteroatoms independently selected from nitrogen, oxygen, or sulfur, wherein the heteroaryl is optionally substituted with halogen, —OR, or —C(O)OR. In some embodiments, a R^{w1} is a 6-membered monocyclic heteroaryl having 1-3 heteroatoms independently selected from nitrogen, oxygen, or sulfur. In some embodiments, a R^{w1} is a 6-membered monocyclic heteroaryl having 1-2 heteroatoms independently selected from nitrogen, oxygen, or sulfur (e.g., pyridyl).

In some embodiments, R^{w1} is as depicted in the compounds of Table 1A or Table 1B, below.

In some embodiments, R^{w1} is hydrogen, methyl, —CF₃, —CH₂C(O)OCH₃,

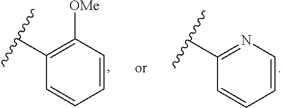 [SMILES: *c1ccccc1OC ; *c1ccccn1]

As described above and defined herein, each R^{x} is independently C₁₋₄ aliphatic, halogen, or —C(O)NR₂. In some embodiments, a R^{x} is C₁₋₄ aliphatic. In some embodiments, a R^{x} is C₁₋₄ alkyl. In some embodiments, a R^{x} is halogen. In some embodiments, a R^{x} is fluoro. In some embodiments, a R^{x} is —C(O)NR₂. In some embodiments, a R^{x} is —C(O)N(H)(R). In some embodiments, R^{x} is —C(O)N(CH₃)₂.

In some embodiments, R is as depicted in the compounds of Table 1A or Table 1B, below.

As described above and defined herein, each R^{y} is independently C₁₋₄ aliphatic, halogen, —OR, or —C(O)NR₂. In some embodiments, a R^{y} is C₁₋₄ aliphatic. In some embodiments, a R^{y} is C₁₋₄ alkyl. In some embodiments, a R^{y} is halogen. In some embodiments, a R^{y} is —OR. In some embodiments, a R^{y} is —C(O)NR₂. In some embodiments, a R^{y} is —C(O)N(H)(R). In some embodiments, R^{y} is —C(O)N(CH₃)₂. In some embodiments, a R^{y} is fluoro, chloro, bromo, iodo, methyl, ethyl, cyclopropyl, —OH, or —OMe.

In some embodiments, R^{y} is as depicted in the compounds of Table 1A or Table 1B, below.

As described above and defined herein, each R is independently hydrogen or C₁₋₄ aliphatic. In some embodiments, each R is independently hydrogen or C₁₋₄ alkyl. In some embodiments, a R is hydrogen. In some embodiments, a R is C₁₋₄ aliphatic. In some embodiments, a R is C₁₋₄ alkyl (e.g., methyl).

In some embodiments, R is as depicted in the compounds of Table 1A or Table 1B, below.

As described above and defined herein, each m is independently 0 or 1. In some embodiments, m is 0. In some embodiments, m is 1.

As described above and defined herein, each n is independently 0, 1, 2, or 3. In some embodiments, n is 0, 1, or 2. In some embodiments, n is 1, 2, or 3. In some embodiments, n is 0 or 1. In some embodiments, n is 1 or 2. In some embodiments, n is 2 or 3. In some embodiments, n is 0. In some embodiments, n is 1. In some embodiments, n is 2. In some embodiments, n is 3.

As described above and defined herein, each of w, x, and y are independently 0, 1, 2, 3, or 4.

In some embodiments, one or more of w, x, and y is 0. In some embodiments, one or more of w, x, and y is 1. In some embodiments, one or more of w, x, and y is 2. In some embodiments, one or more of w, x, and y is 3. In some embodiments, one or more of w, x, and y is 4.

In some embodiments, w is 0, 1, 2, or 3. In some embodiments, w is 0, 1, or 2. In some embodiments, w is 1, 2, or 3. In some embodiments, w is 0 or 1. In some embodiments, w is 1 or 2. In some embodiments, w is 2 or 3. In some embodiments, w is 3 or 4. In some embodiments, w is 0. In some embodiments, w is 1. In some embodiments, w is 2. In some embodiments, w is 3. In some embodiments, w is 4.

In some embodiments, x is 0, 1, 2, or 3. In some embodiments, x is 0, 1, or 2. In some embodiments, x is 1, 2, or 3. In some embodiments, x is 0 or 1. In some embodiments, x is 1 or 2. In some embodiments, x is 2 or 3. In some embodiments, x is 3 or 4. In some embodiments, x is 0. In some embodiments, x is 1. In some embodiments, x is 2. In some embodiments, x is 3. In some embodiments, x is 4.

In some embodiments, y is 0, 1, 2, or 3. In some embodiments, y is 0, 1, or 2. In some embodiments, y is 1, 2, or 3. In some embodiments, y is 0 or 1. In some embodiments, y is 1 or 2. In some embodiments, y is 2 or 3. In some embodiments, y is 3 or 4. In some embodiments, y is 0. In some embodiments, y is 1. In some embodiments, y is 2. In some embodiments, y is 3. In some embodiments, y is 4.

In some embodiments, m, n, w, x, and y are as depicted in the compounds of Table 1A or Table 1B, below.

In some embodiments, the present disclosure provides a compound of formula II-a:

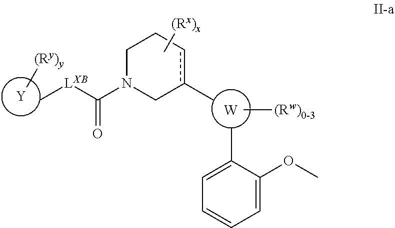

or a pharmaceutically acceptable salt thereof, wherein each variable is as defined above and described herein, both individually and in combination.

In some embodiments, the present disclosure provides a compound of formula III-a:

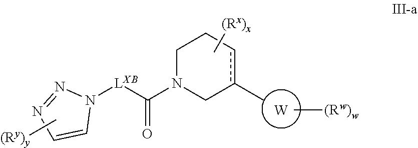

or a pharmaceutically acceptable salt thereof, wherein each variable is as defined above and described herein, both individually and in combination.

In some embodiments, the present disclosure provides a compound of formula IV-a:

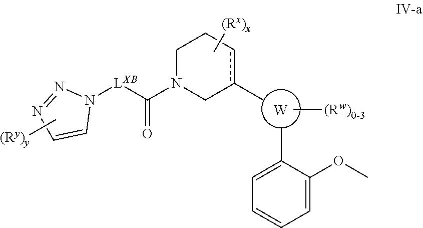

or a pharmaceutically acceptable salt thereof, wherein each variable is as defined above and described herein, both individually and in combination.

In some embodiments, the present disclosure provides a compound of formula I-b-1:

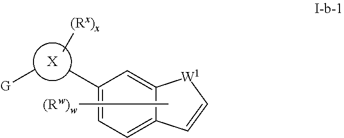

or a pharmaceutically acceptable salt thereof, wherein each variable is as defined above and described herein, both individually and in combination.

In some embodiments, the present disclosure provides a compound of formula I-b-2:

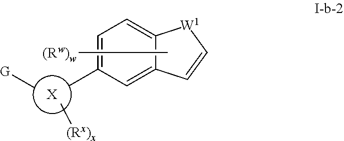

or a pharmaceutically acceptable salt thereof, wherein each variable is as defined above and described herein, both individually and in combination.

In some embodiments, the present disclosure provides a compound of formula II-b:

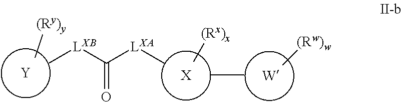

or a pharmaceutically acceptable salt thereof, wherein each variable is as defined above and described herein, both individually and in combination.

In some embodiments, the present disclosure provides a compound of formula II-b-1 or II-b-2:

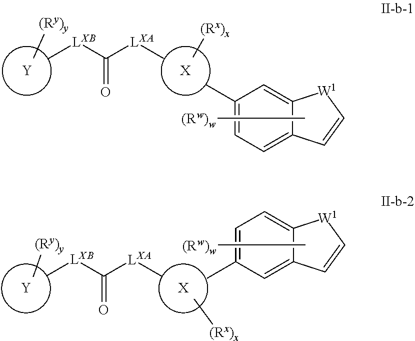

or a pharmaceutically acceptable salt thereof, wherein each variable is as defined above and described herein, both individually and in combination.

In some embodiments, the present disclosure provides a compound of formula III-b:

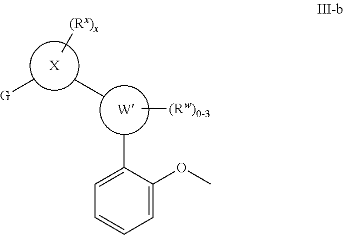

or a pharmaceutically acceptable salt thereof, wherein each variable is as defined above and described herein, both individually and in combination.

In some embodiments, the present disclosure provides a compound of formula III-b-1 or III-b-2:

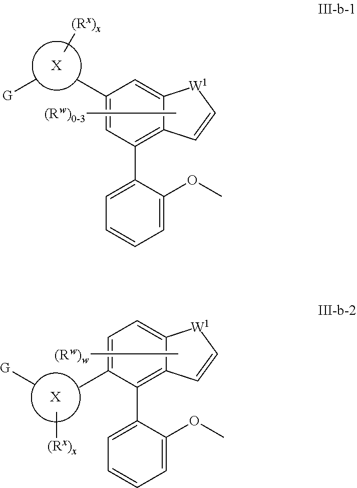

or a pharmaceutically acceptable salt thereof, wherein each variable is as defined above and described herein, both individually and in combination.

In some embodiments, the present disclosure provides a compound of formula IV-b:

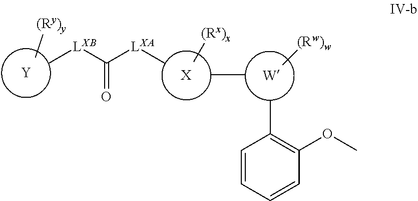

or a pharmaceutically acceptable salt thereof, wherein each variable is as defined above and described herein, both individually and in combination.

In some embodiments, the present disclosure provides a compound of formula TV-b-1 or TV-b-2:

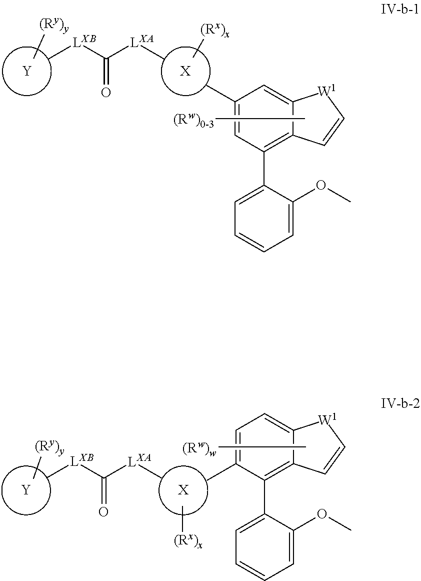

or a pharmaceutically acceptable salt thereof, wherein each variable is as defined above and described herein, both individually and in combination.

In some embodiments, the present disclosure provides a compound of formula V-b:

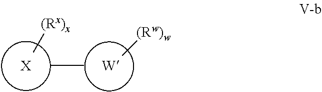

or a pharmaceutically acceptable salt thereof, wherein each variable is as defined above and described herein, both individually and in combination.

In some embodiments, the present disclosure provides a compound of formula V-b-1 or V-b-2:

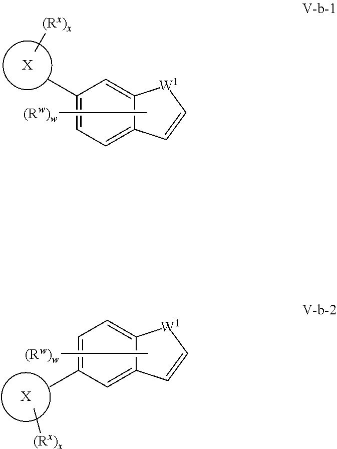

or a pharmaceutically acceptable salt thereof, wherein each variable is as defined above and described herein, both individually and in combination.

In some embodiments, the present disclosure provides a compound of formula V-b-3 or V-b-4:

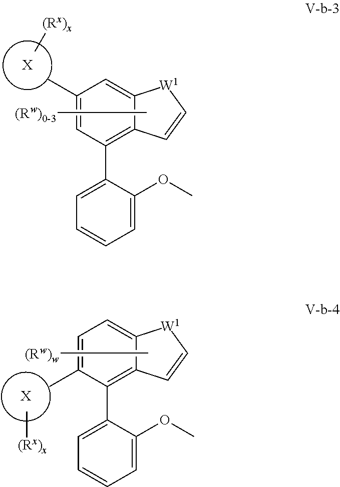

or a pharmaceutically acceptable salt thereof, wherein each variable is as defined above and described herein, both individually and in combination.

In some embodiments, the present disclosure provides a compound of formula VI-b:

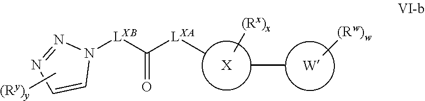

or a pharmaceutically acceptable salt thereof, wherein each variable is as defined above and described herein, both individually and in combination.

In some embodiments, the present disclosure provides a compound of formula VI-b-1 or VI-b-2:

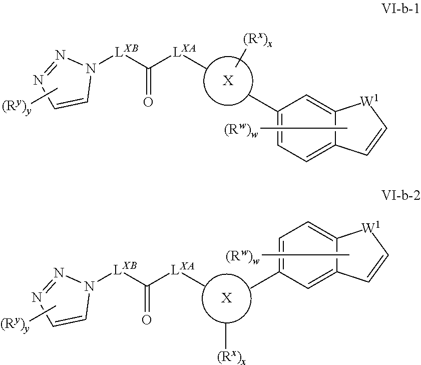

or a pharmaceutically acceptable salt thereof, wherein each variable is as defined above and described herein, both individually and in combination.

In some embodiments, the present disclosure provides a compound of formula VII-b:

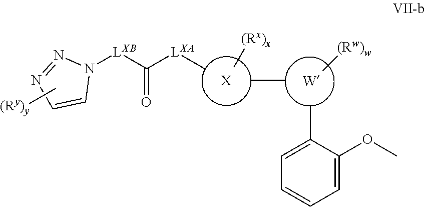

or a pharmaceutically acceptable salt thereof, wherein each variable is as defined above and described herein, both individually and in combination.

In some embodiments, the present disclosure provides a compound of formula VII-b-1 or VII-b-2:

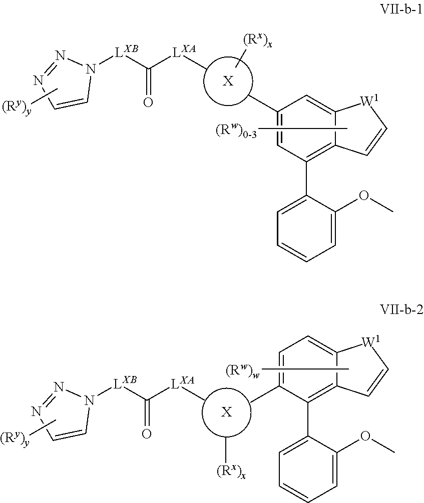

or a pharmaceutically acceptable salt thereof, wherein each variable is as defined above and described herein, both individually and in combination.

Exemplary compounds of the invention are set forth in Table 1A, Table 1B, Table 2A, and Table 2B, below.

TABLE 1A
Exemplary Compounds

| No. | Compound Structure |
|---|---|
| I-6 | 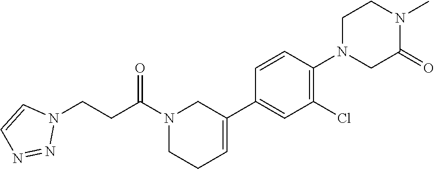 [SMILES: CN1CCN(c2ccc(C3=CCCN(C(=O)CCn4ccnn4)C3)cc2Cl)CC1=O] |
| I-7 | 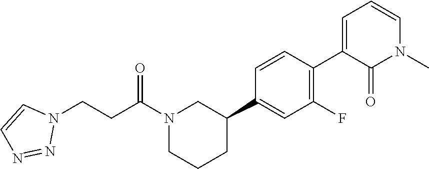 [SMILES: Cn1cccc(-c2ccc([C@H]3CCCN(C(=O)CCn4ccnn4)C3)cc2F)c1=O] |
| I-211 | 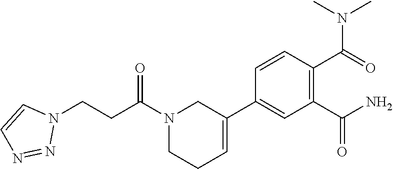 [SMILES: CN(C)C(=O)c1ccc(C2=CCCN(C(=O)CCn3ccnn3)C2)cc1C(N)=O] |
| I-218 | 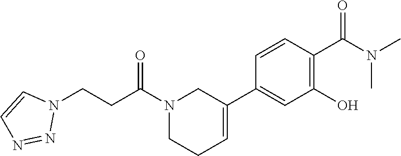 [SMILES: CN(C)C(=O)c1ccc(C2=CCCN(C(=O)CCn3ccnn3)C2)cc1O] |
| I-223 | 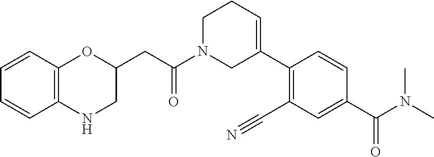 [SMILES: CN(C)C(=O)c1ccc(C2=CCCN(C(=O)CC3CNc4ccccc4O3)C2)c(C#N)c1] |
| I-230 | 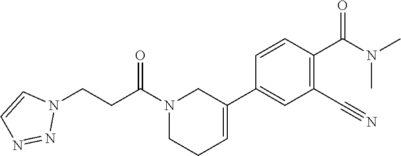 [SMILES: CN(C)C(=O)c1ccc(C2=CCCN(C(=O)CCn3ccnn3)C2)cc1C#N] |
| I-240 | 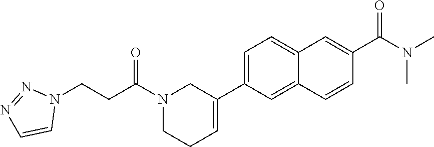 [SMILES: CN(C)C(=O)c1ccc2cc(C3=CCCN(C(=O)CCn4ccnn4)C3)ccc2c1] |
| I-242 | 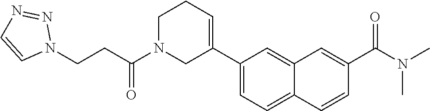 [SMILES: CN(C)C(=O)c1ccc2ccc(C3=CCCN(C(=O)CCn4ccnn4)C3)cc2c1] |
| I-250 | 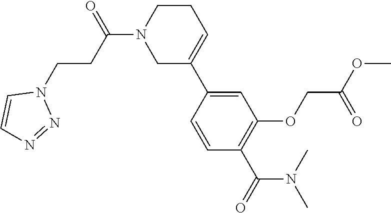 [SMILES: COC(=O)COc1cc(C2=CCCN(C(=O)CCn3ccnn3)C2)ccc1C(=O)N(C)C] |
| I-253 | 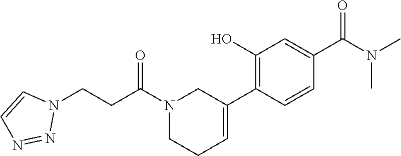 [SMILES: CN(C)C(=O)c1ccc(C2=CCCN(C(=O)CCn3ccnn3)C2)c(O)c1] |
| I-260 | 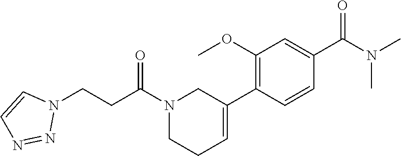 [SMILES: COc1cc(C(=O)N(C)C)ccc1C1=CCCN(C(=O)CCn2ccnn2)C1] |
| I-288 | 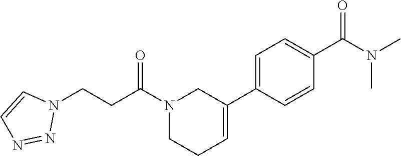 [SMILES: CN(C)C(=O)c1ccc(C2=CCCN(C(=O)CCn3ccnn3)C2)cc1] |
| I-303 | 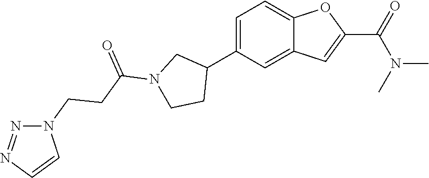 [SMILES: CN(C)C(=O)c1cc2cc(C3CCN(C(=O)CCn4ccnn4)C3)ccc2o1] |
| I-320 | 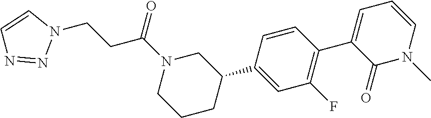 [SMILES: Cn1cccc(-c2ccc([C@@H]3CCCN(C(=O)CCn4ccnn4)C3)cc2F)c1=O] |
| I-836 | 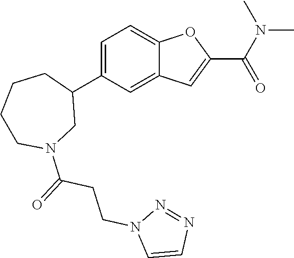 [SMILES: CN(C)C(=O)c1cc2cc(C3CCCCN(C(=O)CCn4ccnn4)C3)ccc2o1] |
| I-1102 | 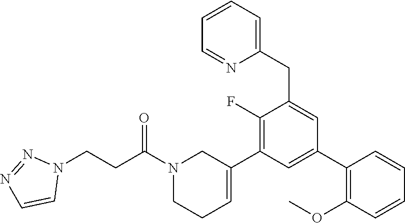 [SMILES: COc1ccccc1-c1cc(Cc2ccccn2)c(F)c(C2=CCCN(C(=O)CCn3ccnn3)C2)c1] |
| I-1293 | 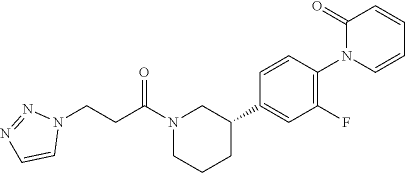 [SMILES: O=C(CCn1ccnn1)N1CCC[C@@H](c2ccc(-n3ccccc3=O)c(F)c2)C1] |
| I-1294 | 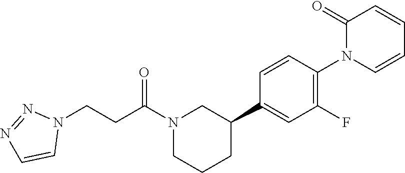 [SMILES: O=C(CCn1ccnn1)N1CCC[C@H](c2ccc(-n3ccccc3=O)c(F)c2)C1] |
| I-1298 | 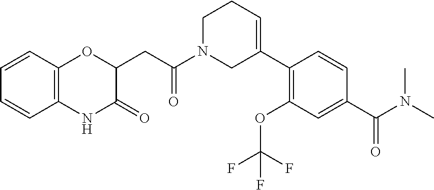 [SMILES: CN(C)C(=O)c1ccc(C2=CCCN(C(=O)CC3Oc4ccccc4NC3=O)C2)c(OC(F)(F)F)c1] |
| I-1323 | 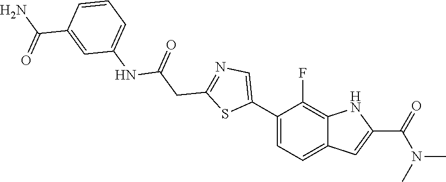 [SMILES: CN(C)C(=O)c1cc2ccc(-c3cnc(CC(=O)Nc4cccc(C(N)=O)c4)s3)c(F)c2[nH]1] |
| I-1325 | 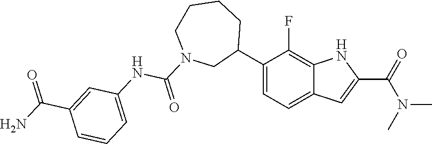 [SMILES: CN(C)C(=O)c1cc2ccc(C3CCCCN(C(=O)Nc4cccc(C(N)=O)c4)C3)c(F)c2[nH]1] |
| I-1583 | 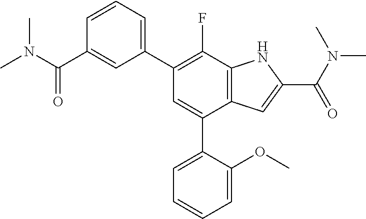 |
| I-1599 | 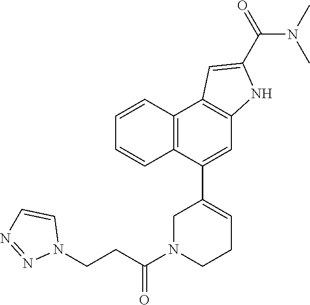 [SMILES: CN(C)C(=O)c1cc2c(cc(C3=CCCN(C(=O)CCn4ccnn4)C3)c3ccccc32)[nH]1] |
| I-1625 | 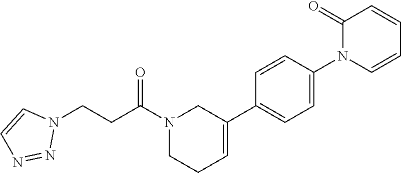 [SMILES: O=C(CCn1ccnn1)N1CCC=C(c2ccc(-n3ccccc3=O)cc2)C1] |

TABLE 1B
Exemplary Compounds

| No. | Compound Structure |
|---|---|
| A1 | 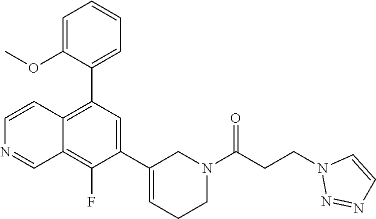 [SMILES: COc1ccccc1-c1cc(C2=CCCN(C(=O)CCn3ccnn3)C2)c(F)c2cnccc12] |
| A2 | 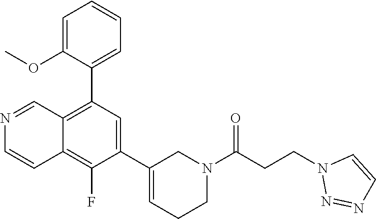 [SMILES: COc1ccccc1-c1cc(C2=CCCN(C(=O)CCn3ccnn3)C2)c(F)c2ccncc12] |
| A3 | 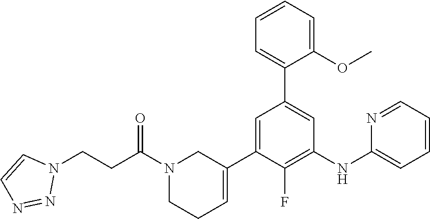 [SMILES: COc1ccccc1-c1cc(Nc2ccccn2)c(F)c(C2=CCCN(C(=O)CCn3ccnn3)C2)c1] |
| A4 | 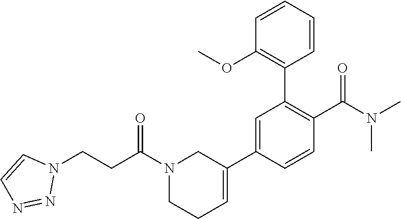 [SMILES: COc1ccccc1-c1cc(C2=CCCN(C(=O)CCn3ccnn3)C2)ccc1C(=O)N(C)C] |
| A5 | 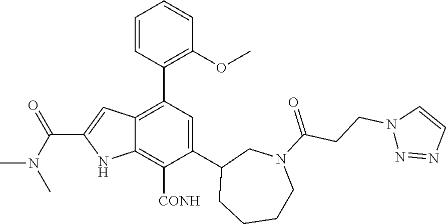 |
| A6 | 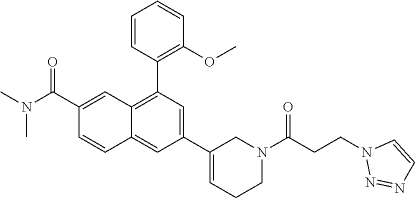 [SMILES: COc1ccccc1-c1cc(C2=CCCN(C(=O)CCn3ccnn3)C2)cc2ccc(C(=O)N(C)C)cc12] |
| A7 | 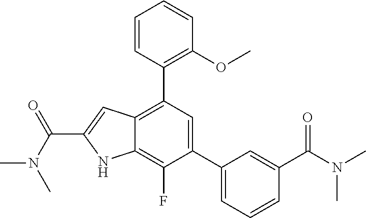 |
| A8 | 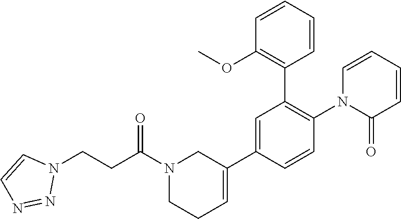 [SMILES: COc1ccccc1-c1cc(C2=CCCN(C(=O)CCn3ccnn3)C2)ccc1-n1ccccc1=O] |
| A9 | 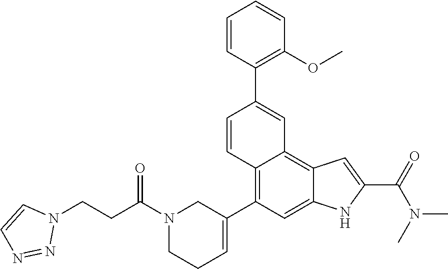 [SMILES: COc1ccccc1-c1ccc2c(C3=CCCN(C(=O)CCn4ccnn4)C3)cc3[nH]c(C(=O)N(C)C)cc3c2c1] |
| A10 | 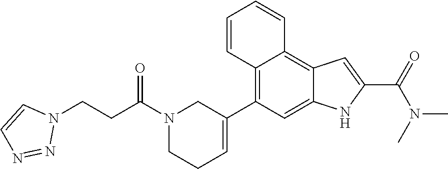 [SMILES: CN(C)C(=O)c1cc2c(cc(C3=CCCN(C(=O)CCn4ccnn4)C3)c3ccccc32)[nH]1] |
| A11 | 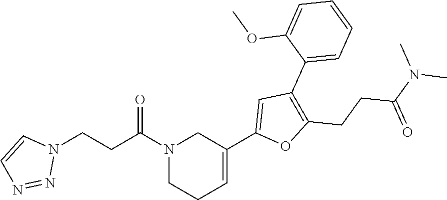 [SMILES: COc1ccccc1-c1cc(C2=CCCN(C(=O)CCn3ccnn3)C2)oc1CCC(=O)N(C)C] |
| A12 | 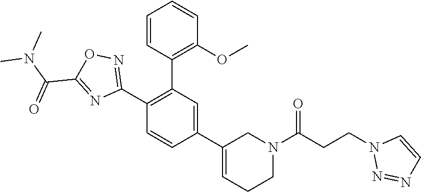 [SMILES: COc1ccccc1-c1cc(C2=CCCN(C(=O)CCn3ccnn3)C2)ccc1-c1noc(C(=O)N(C)C)n1] |
| A13 | 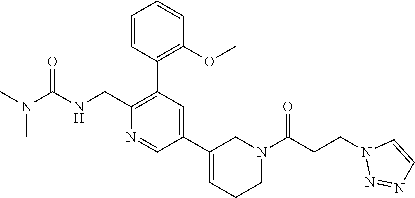 [SMILES: COc1ccccc1-c1cc(C2=CCCN(C(=O)CCn3ccnn3)C2)cnc1CNC(=O)N(C)C] |
| A14 | 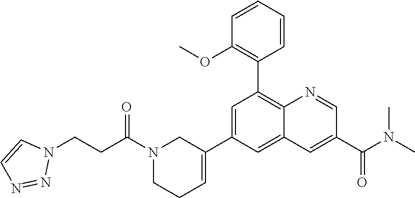 [SMILES: COc1ccccc1-c1cc(C2=CCCN(C(=O)CCn3ccnn3)C2)cc2cc(C(=O)N(C)C)cnc12] |
| A15 | 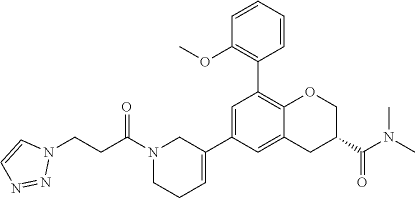 [SMILES: COc1ccccc1-c1cc(C2=CCCN(C(=O)CCn3ccnn3)C2)cc2c1OC[C@H](C(=O)N(C)C)C2] |
| A16 | 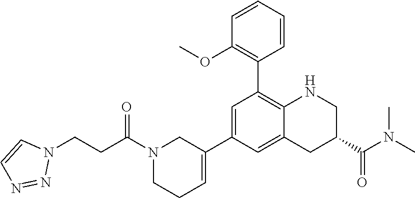 [SMILES: COc1ccccc1-c1cc(C2=CCCN(C(=O)CCn3ccnn3)C2)cc2c1NC[C@H](C(=O)N(C)C)C2] |
| A17 | 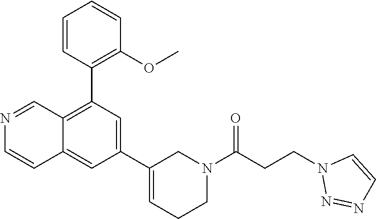 [SMILES: COc1ccccc1-c1cc(C2=CCCN(C(=O)CCn3ccnn3)C2)cc2ccncc12] |
| A18 | 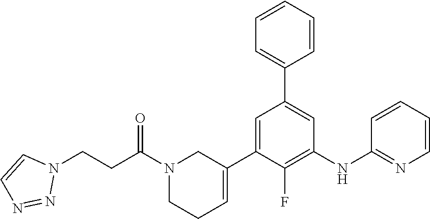 [SMILES: O=C(CCn1ccnn1)N1CCC=C(c2cc(-c3ccccc3)cc(Nc3ccccn3)c2F)C1] |
| A19 | 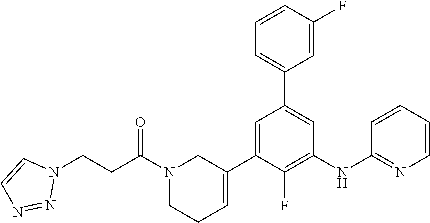 [SMILES: O=C(CCn1ccnn1)N1CCC=C(c2cc(-c3cccc(F)c3)cc(Nc3ccccn3)c2F)C1] |
| A20 | 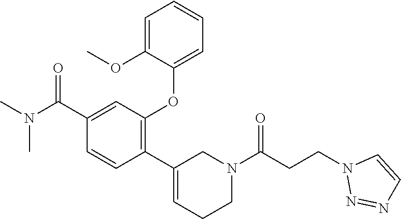 [SMILES: COc1ccccc1Oc1cc(C(=O)N(C)C)ccc1C1=CCCN(C(=O)CCn2ccnn2)C1] |
| A21 | 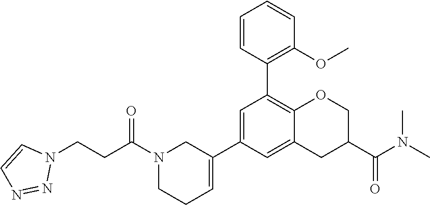 [SMILES: COc1ccccc1-c1cc(C2=CCCN(C(=O)CCn3ccnn3)C2)cc2c1OCC(C(=O)N(C)C)C2] |
| A22 | 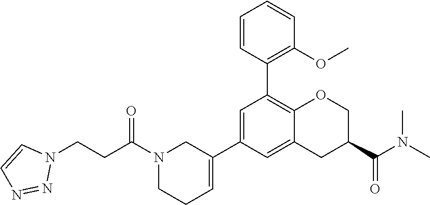 [SMILES: COc1ccccc1-c1cc(C2=CCCN(C(=O)CCn3ccnn3)C2)cc2c1OC[C@@H](C(=O)N(C)C)C2] |

TABLE 2A
Exemplary Compounds

| No. | Compound Structure |
|---|---|
| I-121 | 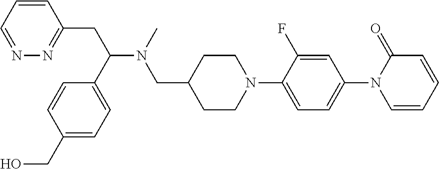 [SMILES: CN(CC1CCN(c2ccc(-n3ccccc3=O)cc2F)CC1)C(Cc1cccnn1)c1ccc(CO)cc1] |
| I-131 | 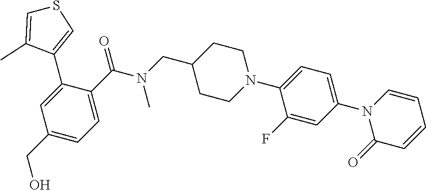 [SMILES: Cc1cscc1-c1cc(CO)ccc1C(=O)N(C)CC1CCN(c2ccc(-n3ccccc3=O)cc2F)CC1] |
| I-164 | 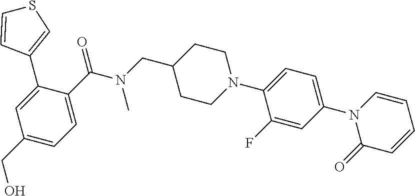 [SMILES: CN(CC1CCN(c2ccc(-n3ccccc3=O)cc2F)CC1)C(=O)c1ccc(CO)cc1-c1ccsc1] |
| I-183 | 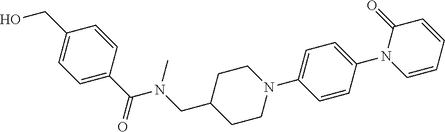 [SMILES: CN(CC1CCN(c2ccc(-n3ccccc3=O)cc2)CC1)C(=O)c1ccc(CO)cc1] |
| I-238 | 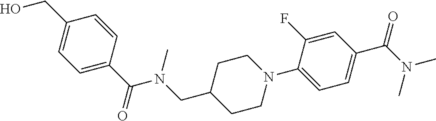 [SMILES: CN(C)C(=O)c1ccc(N2CCC(CN(C)C(=O)c3ccc(CO)cc3)CC2)c(F)c1] |
| I-258 | 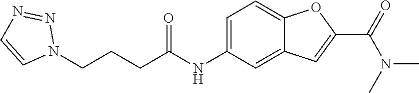 [SMILES: CN(C)C(=O)c1cc2cc(NC(=O)CCCn3ccnn3)ccc2o1] |
| I-262 | 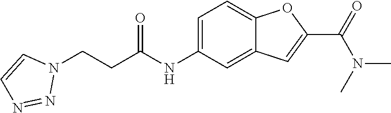 [SMILES: CN(C)C(=O)c1cc2cc(NC(=O)CCn3ccnn3)ccc2o1] |
| I-1240 | 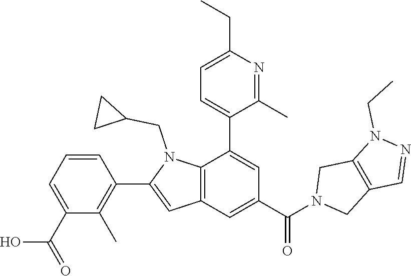 [SMILES: CCc1ccc(-c2cc(C(=O)N3Cc4cnn(CC)c4C3)cc3cc(-c4cccc(C(=O)O)c4C)n(CC4CC4)c23)c(C)n1] |
| I-1246 | 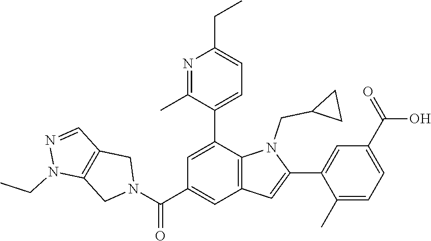 [SMILES: CCc1ccc(-c2cc(C(=O)N3Cc4cnn(CC)c4C3)cc3cc(-c4cc(C(=O)O)ccc4C)n(CC4CC4)c23)c(C)n1] |
| I-1271 | 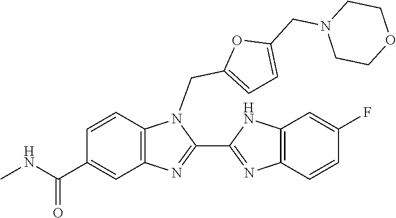 [SMILES: CNC(=O)c1ccc2c(c1)nc(-c1nc3ccc(F)cc3[nH]1)n2Cc1ccc(CN2CCOCC2)o1] |
| I-1272 | 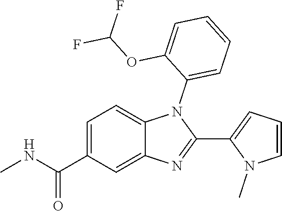 [SMILES: CNC(=O)c1ccc2c(c1)nc(-c1cccn1C)n2-c1ccccc1OC(F)F] |
| I-1273 | 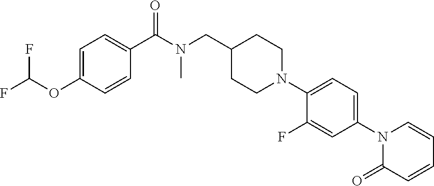 [SMILES: CN(CC1CCN(c2ccc(-n3ccccc3=O)cc2F)CC1)C(=O)c1ccc(OC(F)F)cc1] |
| I-1295 | 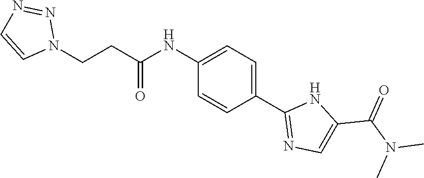 [SMILES: CN(C)C(=O)c1cnc(-c2ccc(NC(=O)CCn3ccnn3)cc2)[nH]1] |
| I-1296 | 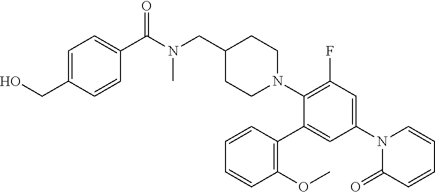 [SMILES: COc1ccccc1-c1cc(-n2ccccc2=O)cc(F)c1N1CCC(CN(C)C(=O)c2ccc(CO)cc2)CC1] |
| I-1297 | 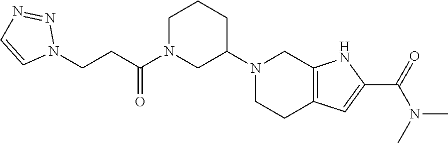 [SMILES: CN(C)C(=O)c1cc2c([nH]1)CN(C1CCCN(C(=O)CCn3ccnn3)C1)CC2] |

TABLE 2B
Exemplary Compounds

| No. | Compound Structure |
|---|---|
| B1 | 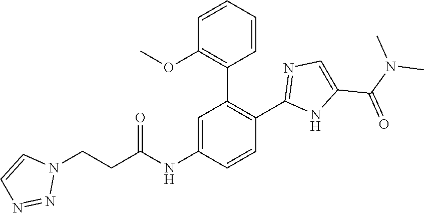 [SMILES: COc1ccccc1-c1cc(NC(=O)CCn2ccnn2)ccc1-c1ncc(C(=O)N(C)C)[nH]1] |
| A23 | 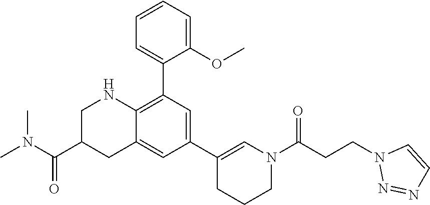 [SMILES: COc1ccccc1-c1cc(C2=CN(C(=O)CCn3ccnn3)CCC2)cc2c1NCC(C(=O)N(C)C)C2] |

In some embodiments, the present invention provides a compound set forth in Table 1A, above, or a pharmaceutically acceptable salt thereof. In some embodiments, the present invention provides a compound set forth in Table 1A, above. In some embodiments, the present invention provides a compound set forth in Table 1B, above, or a pharmaceutically acceptable salt thereof. In some embodiments, the present invention provides a compound set forth in Table 1B, above. In some embodiments, the present invention provides a compound set forth in Table 2A, above, or a pharmaceutically acceptable salt thereof. In some embodiments, the present invention provides a compound set forth in Table 2A, above. In some embodiments, the present invention provides a compound set forth in Table 2B, above, or a pharmaceutically acceptable salt thereof. In some embodiments, the present invention provides a compound set forth in Table 2B, above.

In some embodiments, the present invention provides a pharmaceutical composition comprising a compound disclosed herein (described in embodiments herein, both singly and in combination), or a pharmaceutically acceptable salt thereof, together with a pharmaceutically acceptable carrier, adjuvant, or vehicle. For example, in some embodiments, the present invention provides a pharmaceutical composition comprising a provided compound (e.g., a compound of formula I-a or I-b as defined above), or a pharmaceutically acceptable salt thereof, together with a pharmaceutically acceptable carrier, adjuvant, or vehicle. In some embodiments, the present invention provides a pharmaceutical composition comprising a provided compound (e.g., a compound of formula I-a or I-b as defined above), together with a pharmaceutically acceptable carrier, adjuvant, or vehicle. In some embodiments, the present invention provides a pharmaceutical composition comprising a compound set forth in Table 1A above, or a pharmaceutically acceptable salt thereof, together with a pharmaceutically acceptable carrier, adjuvant, or vehicle. In some embodiments, the present invention provides a pharmaceutical composition comprising a compound set forth in Table 1A above, together with a pharmaceutically acceptable carrier, adjuvant, or vehicle. In some embodiments, the present invention provides a pharmaceutical composition comprising a compound set forth in Table 1B above, or a pharmaceutically acceptable salt thereof, together with a pharmaceutically acceptable carrier, adjuvant, or vehicle. In some embodiments, the present invention provides a pharmaceutical composition comprising a compound set forth in Table 1B above, together with a pharmaceutically acceptable carrier, adjuvant, or vehicle. In some embodiments, the present invention provides a pharmaceutical composition comprising a compound set forth in Table 2A above, or a pharmaceutically acceptable salt thereof, together with a pharmaceutically acceptable carrier, adjuvant, or vehicle. In some embodiments, the present invention provides a pharmaceutical composition comprising a compound set forth in Table 2A above, together with a pharmaceutically acceptable carrier, adjuvant, or vehicle. In some embodiments, the present invention provides a pharmaceutical composition comprising a compound set forth in Table 2B above, or a pharmaceutically acceptable salt thereof, together with a pharmaceutically acceptable carrier, adjuvant, or vehicle. In some embodiments, the present invention provides a pharmaceutical composition comprising a compound set forth in Table 2B above, together with a pharmaceutically acceptable carrier, adjuvant, or vehicle.

In some embodiments, the present invention provides a provided compound (e.g., a compound of formula I-a or I-b as defined above), or a pharmaceutically acceptable salt thereof, or a pharmaceutical composition comprising a provided compound (e.g., a compound of formula I-a or I-b as defined above), or a pharmaceutically acceptable salt thereof, and a pharmaceutically acceptable carrier, adjuvant, or vehicle for use as a medicament.

In some embodiments, the invention also provides a compound described herein (such as a compound of formula I-a or I-b), or pharmaceutical compositions described herein, for use in a method for modulating (e.g., inhibiting) STAT6 as described herein. In some embodiments, the invention also provides a compound described herein (such as a compound of formula I-a or I-b), or pharmaceutical compositions described herein, for use in a method for treating a STAT6-mediated disorder as described herein.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably modulate (e.g., inhibit) STAT6 protein, or a mutant thereof, in a biological sample or in a patient. In certain embodiments, the amount of compound in compositions of this invention is such that is effective to measurably modulate (e.g., inhibit) STAT6 protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or prodrug of a compound of this invention that, upon administration to a recipient, is capable of providing, either directly or indirectly, a compound of this invention or an inhibitory active metabolite or residue thereof. In some embodiments, a “pharmaceutically acceptable derivative” is a pharmaceutically acceptable salt.

As used herein, the term “inhibitory active metabolite or residue thereof” means that a metabolite or residue thereof is also an inhibitor of STAT6 protein, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the modulation of STAT6 protein activity including phosphorylated or activated STAT6 protein (e.g., pSTAT6) activity.

According to one embodiment, the invention relates to a method of modulating (e.g., inhibiting) STAT6 or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. In some embodiments, the STAT6 is from a biological sample. In some embodiments, the biological sample is taken from a subject.

Inhibition of STAT6, or a mutant thereof, activity in a biological sample is useful for a variety of purposes that are known to one of skill in the art. Examples of such purposes include, but are not limited to, blood transfusion, organ-transplantation, biological specimen storage, and biological assays.

According to another embodiment, the invention relates to a method of modulating (e.g., inhibiting) STAT6, or a mutant thereof, activity in a patient comprising the step of administering to said patient a compound of the present invention, or a composition comprising said compound. In other embodiments, the present invention provides a method for treating a disorder mediated by STAT6 or a mutant thereof, in a patient in need thereof, comprising the step of administering to said patient a compound according to the present invention or pharmaceutically acceptable composition thereof. Such disorders are described in detail herein.

The activity of a compound utilized in this invention as a modulator (e.g., inhibitor) of STAT6 or a mutant thereof, may be assayed in vitro, in vivo or in a cell line. In vitro assays include assays that determine inhibition of either the activity and/or the subsequent functional consequences of activated STAT6 protein or a mutant thereof. Alternate in vitro assays quantitate the ability of the inhibitor to bind to STAT6 protein. Inhibitor binding may be measured by radiolabeling the inhibitor prior to binding, isolating the inhibitor/STAT6 complex and determining the amount of radiolabel bound. Alternatively, inhibitor binding may be determined by running a competition experiment where new inhibitors are incubated with STAT6 protein bound to known radioligands. Detailed conditions for assaying a compound utilized in this invention as a modulator (e.g., inhibitor) of STAT proteins, or a mutant thereof, are set forth in the Examples below.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are modulators (e.g., inhibitors) of STAT6 protein and are therefore useful for treating one or more disorders associated with activity of STAT6 protein. Thus, in certain embodiments, the present invention provides a method for treating a STAT6-mediated disorder comprising the step of administering to a patient in need thereof a compound of the present invention, or pharmaceutically acceptable composition thereof.

As used herein, the term “STAT6-mediated” disorders, diseases, and/or conditions as used herein means any disease or other deleterious condition in which STAT6 or a mutant thereof, are known to play a role. Accordingly, another embodiment of the present invention relates to treating or lessening the severity of one or more diseases in which STAT6 or a mutant thereof, are known to play a role.

STAT6 functions as a transcription factor to induce gene expression and plays an important role in the IL-4/IL-13 signaling pathway and thus is critical in IL-4/IL-13 mediated biological responses including in human malignancies (e.g., Patel, B. K. R., et al. “Localization of the human stat6 gene to chromosome 12q13. 3-q14. 1, a region implicated in multiple solid tumors.” Genomics 52.2 (1998): 192-200). The STAT6-mediated signaling pathway has been shown to be required for the development of T-helper type 2 (Th2) cells and Th2 immune response and plays a critical role in Th2 lung inflammatory responses including clearance of parasitic infections and in the pathogenesis of asthma (e.g., Walford, H. H. and Doherty, T. A “STAT6 and lung inflammation.”Jak-stat 2.4 (2013): e25301). It has been found that STAT6 induces the expression of BCL2L1/BCL-X(L), which is responsible for the anti-apoptotic activity of IL-4 and is shown to play a prominent role in adaptive immunity such as providing innate immune signaling in response to virus infection (e.g., Chen, H., et al. “Activation of STAT6 by STING is critical for antiviral innate immunity.” Cell 147.2 (2011): 436-446). Knockout studies in mice have suggested the role STAT6 in differentiation of T helper 2 (Th2), expression of cell surface markers, and class switch of immunoglobulins. STAT6 protein also regulates other transcription factor as Gata3, which is important regulator of Th2 differentiation. STAT6 is also required for the development of IL-9-secreting T cells.

In some embodiments, biomarkers associated with the IL-4/13 pathway include IgE, Thymus and activation regulated chemokine (TARC), CD23, periostin, and eisinophils. TARC is a serum TH2 biomarker and chemoattractant for TH2 cell. CD23 is a B cell activation marker and correlates with IgE class switch. Periostin is a serum TH2 biomarker and ECM protein associated with tissue remodeling in atopic diseases.

In some embodiments, treatment with a provided compound results in lesser IL-4 induced TARC release compared to a reference or standard level. In some embodiments, treatment with a provided compound results in lesser IL-13 induced CD23 expression compared to a reference or standard level. In some embodiments, treatment with a provided compound results in lesser IL-13 induced periostin release compared to a reference or standard level.

In some embodiments, treatment with a provided compound inhibits IL-4 induced TARC release. In some embodiments, treatment with a provided compound inhibits IL-13 induced CD23 expression. ISE, treatment with a provided compound inhibits IL-13 induced periostin release.

In some embodiments, the present invention provides a method for treating one or more disorders, diseases, and/or conditions wherein the disorder, disease, or condition is a neurodegenerative disorder, a viral disease, an autoimmune disease, an inflammatory disorder, conditions associated with organ transplantation, immunodeficiency disorders, an infectious disease, thrombin-induced platelet aggregation, liver disease, or pathologic immune conditions involving T cell activation.

Diseases and conditions treatable according to the methods of this invention include, but are not limited to, viral disease, autoimmune diseases, autoinflammatory syndromes, atherosclerosis, psoriasis, allergic disorders, inflammatory bowel disease, inflammation, acute and chronic gout and gouty arthritis, neurological disorders, immunodeficiency disorders such as AIDS and HIV, osteoarthritis, infectious diseases, and pathologic immune conditions involving T cell activation in a patient. In one embodiment, a human patient is treated with a compound of the current invention and a pharmaceutically acceptable carrier, adjuvant, or vehicle, wherein said compound is present in an amount to measurably modulate (e.g., inhibit) STAT6 or a mutant thereof

Compounds according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma exacerbated or induced following bacterial or viral infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics.

Another aspect of the present invention relates to a method of treating an allergic or inflammatory disease in a subject comprising administering to the subject a therapeutically effective amount of a compound of the present invention to the subject. The disease may be a lung disease such as, e.g., asthma, airway hyperresponsiveness (AHR), an allergic disease, allergic rhinitis, emphysema, chronic obstructive pulmonary disease (COPD), reactive airway disease, chronic rhinosinusitis, or essentially any other disease of the upper or lower airways that produces airflow obstruction.

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example antiinflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterized by asthma attack, e.g., between the hours of about 4 to 6 am, i.e., at a time normally substantially distant from any previously administered symptomatic asthma therapy.

In some embodiments, STAT6, via its Src homology 2 (SH2) domain, is recruited to the phosphotyrosine residues and is phosphorylated on Tyr641. In some embodiments, STAT6 then dimerizes via reciprocal SH2 domain-pTyr641 interactions, translocates to the nucleus, and participates in the expression of genes leading to asthma and airway hyperresponsiveness (AHR).

In some embodiments, the present invention provides a method of treating asthma in a patient in need thereof, comprising administering a compound of the present invention, or a pharmaceutically acceptable salt thereof.

In some embodiments, the present invention provides a method of treating airway hyperresponsiveness (AHR) in a patient in need thereof, comprising administering a compound of the present invention, or a pharmaceutically acceptable salt thereof.

In some embodiments, the present invention provides a method of treating allergic rhinitis in a patient in need thereof, comprising administering a compound of the present invention, or a pharmaceutically acceptable salt thereof.

In some embodiments, the present invention provides a method of treating allergic asthma in a patient in need thereof, comprising administering a compound of the present invention, or a pharmaceutically acceptable salt thereof.

In some embodiments, the present invention provides a method of treating emphysema in a patient in need thereof, comprising administering a compound of the present invention, or a pharmaceutically acceptable salt thereof.

In some embodiments, the present invention provides a method of treating chronic rhinosinusitis in a patient in need thereof, comprising administering a compound of the present invention, or a pharmaceutically acceptable salt thereof.

In some embodiments, the present invention provides a method of treating COPD in a patient in need thereof, comprising administering a compound of the present invention, or a pharmaceutically acceptable salt thereof.

Compounds of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activation, compounds of the invention are also useful in the treatment of eosinophil related disorders, e.g., eosinophilia, in particular eosinophil related disorders of the airways (e.g., involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of the invention are also useful in the treatment of inflammatory or allergic conditions of the skin. In some embodiments, the present invention provides a method of treating inflammatory or allergic conditions of the skin in a patient in need thereof, comprising administering a compound of the present invention, or a pharmaceutically acceptable salt thereof.

In some embodiments the inflammatory disease of the skin is selected from psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis or primary biliary cholangitis, uveitis (anterior and posterior), Sjogren's syndrome, keratoconjunctivitis sicca and vernal keratoconjunctivitis, interstitial lung disease or fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, encephalomyelitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, the present invention provides a method of treating an autoimmune disease selected from encephalomyelitis, systemic sclerosis, idiopathic pulmonary fibrosis (IPF), inflammatory bowel disease, atopic dermatitis, rheumatoid arthritis, graft versus host disease (acute and chronic), and other tissue fibrosis diseases.

In some embodiments, the present invention provides a method of treating idiopathic interstitial pneumonia(s) (IIPs), including any type of lung fibrosis, either interstitial lung disease associated with rheumatic disease (including SSc) or IPF itself, in a patient in need thereof, comprising administering a compound of the present invention, or a pharmaceutically acceptable salt thereof.

In some embodiments, the inflammatory disease which can be treated according to the methods of this invention is selected from acute and chronic gout, chronic gouty arthritis, psoriasis, psoriatic arthritis, rheumatoid arthritis, Juvenile rheumatoid arthritis, systemic juvenile idiopathic arthritis (SJIA), cryopyrin associated periodic syndrome (CAPS), and osteoarthritis.

In some embodiments, the inflammatory disease which can be treated according to the methods of this invention is a TH17 mediated disease or TH17-associated disease. In some embodiments, the TH17 mediated disease or TH17-associated disease is selected from psoriasis, psoriatric arthritis, systemic lupus erythematosus, multiple sclerosis, and inflammatory bowel disease (including Crohn's disease or ulcerative colitis), or graft-versus-host disease.

In some embodiments, the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

In some embodiments, the present invention provides a method of treating an autoimmune disease or inflammatory disorder is selected from nonalcoholic fatty liver disease (NAFLD), nonalcoholic steatohepatitis (NASH), idiopathic autoimmune hepatitis, progressive fibrosis associated interstitial lung disease, pulmonary arterial hypertension (PAH), immunoglobulin G4-related disease (IgG4-RD), chronic organ rejection (e.g., lung transplant), vasculitides (e.g., vasculitides), and STAT6 gain of function (GOF) mutations.

In some embodiments, the present invention provides a method of treating STAT6 gain of function (GOF) mutations in a patient in need thereof, comprising administering a compound of the present invention, or a pharmaceutically acceptable salt thereof. In some embodiments, the STAT6 GOF mutation is STAT6VT.

In some embodiments, the cardiovascular disease which can be treated according to the methods of the present invention include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the methods of the present invention include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In some embodiments, the invention provides a method of treating, preventing or lessening the severity of Alzheimer's disease comprising administering to a patient in need thereof a provided compound or a pharmaceutically acceptable salt or composition thereof.

In some embodiments, the invention provides a method of treating a disease or condition commonly occurring in connection with transplantation. In some embodiments, the disease or condition commonly occurring in connection with transplantation is selected from organ transplantation, organ transplant rejection, and graft versus host disease.

In some embodiments, the invention provides a method of treating a metabolic disease. In some embodiments the metabolic disease is selected from Type 1 diabetes, Type 2 diabetes, metabolic syndrome, and obesity.

In some embodiments, the invention provides a method of treating a viral disease. In some embodiments, the viral infection is HIV or COVID19 infection.

In some embodiments, the present invention provides a method of treating a JAK-associated disease other than cancer.

Furthermore, the invention provides the use of a compound according to the definitions herein, or a pharmaceutically acceptable salt, or a hydrate or solvate thereof for the preparation of a medicament for the treatment of an inflammatory disease, an obstructive respiratory disease, a neurodegenerative disease, a viral disease, or a disorder commonly occurring in connection with transplantation.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent. It will be appreciated that the present disclosure contemplates use of the combination therapies described herein only for treating the diseases, disorders, and conditions described herein.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif©), glatiramer acetate (Copaxone®), and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids agents that prolong or improve pharmacokinetics such as cytochrome P450 inhibitors (i.e., inhibitors of metabolic breakdown) and CYP3A4 inhibitors (e.g., ketokenozole and ritonavir), pirfenidone (Esbriet®), nintedanib (Ofev®), intravenous immunoglobulins, bosentan (Tracleer®), nifedipine (Procardia XL®), sildenafil (Revatio®), losartan (Cozaar®), iloprost (Ventavis®), topical nitroglycerin, N-acetylcysteine, antiacid therapy, and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-Bid®, Uniphyl®, Theo-24R) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-Bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-Bid®, Uniphyl®, Theo-24@R) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®.

In some embodiments, the present invention provides a method of treating HIV comprising administering to a patient in need thereof a provided compound and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells. Other approved therapeutics that inhibit bone resorption include bisphosphonates, such as zoledronic acid (Zometa®, Novartis).

In some embodiments, the present invention provides a method of treating Alzheimer's disease comprising administering to a patient in need thereof a provided compound and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, diseases of the bone and joints leading to joint inflammation and pain, cartilage and/or bone destruction, as well as bone regrowth and fusion, including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, idiopathic autoimmune hepatitis, gastritis, enteritis, dermatitis, gingivitis, appendicitis, pancreatitis, cholecystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, myasthenia gravis, Hashimoto's thyroiditis, atopic dermatitis, degenerative joint disease, vitiligo, autoimmune hypopituitarism, Guillain-Barre syndrome, Behcet's disease, scleroderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a neurodegenerative disorder, an angiogenic disorder, a viral disease, an autoimmune disease, an inflammatory disorder, conditions associated with organ transplantation, immunodeficiency disorders, an infectious disease, thrombin-induced platelet aggregation, liver disease, pathologic immune conditions involving T cell activation, and a cardiovascular disorder.

In another embodiment, the present invention provides a method of treating or lessening the severity of a disease comprising administering to a patient in need thereof a provided compound and a PI3K inhibitor, wherein the disease is selected from psoriasis or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced or exacerbated following bacterial or viral infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung disease or fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™.

The term “anti-androgen” as used herein relates to any substance which is capable of inhibiting the biological effects of androgenic hormones and includes, but is not limited to, bicalutamide (Casodex™) The term “gonadorelin agonist” as used herein includes, but is not limited to abarelix, goserelin and goserelin acetate. Goserelin can be administered under the trade name Zoladex™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further anti-angiogenic compounds include compounds having another mechanism for their activity, e.g. unrelated to protein or lipid kinase inhibition e.g. thalidomide (Thalomid™) and TNP-470.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

Also included are EDG binders and ribonucleotide reductase inhibitors. The term “EDG binders” as used herein refers to a class of immunosuppressants that modulates lymphocyte recirculation, such as FTY720.

The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vemalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH—55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 g/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### EXEMPLIFICATION

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

TABLE 3
Analytical instruments

| | |
|---|---|
| LCMS | Shimadzu UFLC MS: LCMS-2020 |
| | Agilent Technologies 1200 series MS: Agilent Technologies 6110 |
| | Agilent Technologies 1200 series MS: LC/MSD VL |
| NMR | BRUKER AVANCE III/400; Frequency (MHz) 400.13; Nucleus: 1H; |
| | Number of Transients: 8 |
| Prep-HPLC | Gilson GX-281 systems: instruments GX-A, GX-B, GX-C, GX-D, |
| | GX-E, GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH+] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH+] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH3·H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19) mm, 5μ. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

In some instances, intermediates and compounds described in the examples comprise one or more stereocenters and more than one enantiomer/diastereomer was produced. In some embodiments, these enantiomers/diastereomers were separated and isolated, although stereochemistry was not resolved. Unless otherwise stated, stereochemistry was assigned arbitrarily. For intermediates, each enantiomer/diastereomer with arbitrarily assigned stereochemistry may result in a final compound (e.g., assigned a “I—” number), which also maintains the arbitrarily assigned stereochemistry. Accordingly, any compound with arbitrarily assigned stereochemistry or produced from an intermediate with arbitrarily assigned stereochemistry may be depicted herein as a certain stereoisomer, but it is understood that such compound may be the other stereoisomer (i.e., enantiomer or diastereomer).

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

TABLE 4
Compound synthesized via coupling of corresponding amines and carboxylic acids

| | LCMS | |
|---|---|---|
| | (ESI+) | |
| | m/z | |
| I-# | (M + H)⁺ | ¹H NMR (400 MHz, DMSO-d₆) δ |
| I-836 | 410.2 | 8.15-8.07 (m, 1H), 7.70 (dd, J = 0.8, 3.6 Hz, 1H), 7.64-7.57 (m, 2H), 7.40- |
| | | 7.32 (m, 2H), 4.66-4.60 (m, 2H), 4.03-3.86 (m, 1H), 3.75-3.62 (m, 1H), 3.48- |
| | | 3.36 (m, 1H), 3.28-3.21 (m, 3H), 3.21-3.10 (m, 2H), 3.06 (s, 1H), 3.05-3.00 |
| | | (m, 3H), 2.92-2.86 (m, 1H), 1.90-1.64 (m, 5H), 1.38-1.24 (m, 1H) |

TABLE 5
Compounds synthesized via coupling of corresponding halogens and boronic acids

| | LCMS | |
|---|---|---|
| | (ESI+) m/z | |
| I-# | (M + H)⁺ | ¹H NMR (400 MHz, DMSO-d₆) δ |
| I- | 499.4 | 2.2-2.3 (m, 2 H), 3.1-3.1 (m, 2 H), 3.6-3.7 (m, 2 H), 3.8 (br s, 3 H), 4.3- |
| 1102 | | 4.3 (m, 2 H), 4.6-4.6 (m, 2 H), 6.1-6.2 (m, 1 H), 6.7-6.8 (m, 1 H), 6.9-7.0 |
| | | (m, 3 H), 7.1-7.1 (m, 1 H), 7.3-7.4 (m, 2 H), 7.5-7.6 (m, 1 H), 7.7-7.7 (m, |
| | | 1 H), 8.1 (br d, J = 7.6 Hz, 2 H), 8.1-8.2 (m, 1 H), 8.7-8.7 (m, 1 H) |
| I- | 588.2 | 7.97-7.90 (m, 2H), 7.86 (s, 1H), 7.78 (d, J = 7.6 Hz, 1H), 7.46 (d, J = 8.0 Hz, |
| 1246¹ | | 1H), 7.32-7.19 (m, 2H), 7.16 (d, J = 1.2 Hz, 1H), 6.71 (s, 1H), 4.88-4.77 |
| | | (m, 2H), 4.62 (d, J = 11.6 Hz, 2H), 4.15-4.08 (m, 1H), 4.06-3.99 (m, 1H), |
| | | 2.84-2.77 (m, 2H), 2.55 (s, 3H), 2.29-2.22 (m, 5H), 1.40-1.24 (m, 6H), |
| | | 0.45-0.27 (m, 1H), 0.03-−0.07 (m, 2H), −0.41-−0.59 (m, 1H), −0.65-−0.77 |
| | | (m, 1H) |

¹The product of the coupling was further hydrolyzed with LiOH in THF at rt for 1 hr.

TABLE 6
Compound synthesized via other standard methodology.

| | LCMS | |
|---|---|---|
| | (ESI+) | |
| | m/z | |
| I-# | (M + H)+ | ¹H NMR (400 MHz, DMSO-d₆) δ |
| I- | 588.5 | 8.66 (br d, J = 7.2 Hz, 1H), 8.05 (s, 1H), 7.90 (dd, J = 6.8, 19.6 Hz, 2H), 7.55 |
| 1240 | | (s, 1H), 7.42 (d, J = 7.6 Hz, 1H), 7.37-7.14 (m, 2H), 6.75 (s, 1H), 4.86-4.80 (m, |
| | | 2H), 4.61 (s, 2H), 4.15-4.00 (m, 2H), 3.35 (s, 1H), 3.20 (d, J = 7.6 Hz, 2H), |
| | | 3.16-2.90 (m, 1H), 2.65 (d, J = 6.6 Hz, 1H), 2.54 (d, J = 7.2 Hz, 2H), 2.42- |
| | | 2.27 (m, 3H), 1.42-1.25 (m, 6H), 0.48-0.23 (m, 1H), 0.05 (s, 2H), −0.34-−0.84 |
| | | (m, 2H) |

### Example 1. Homogenous Time-Resolved Fluorescence (HTRF) Binding Assay

HTRF binding assays were performed using 0.15 nM biotinylated truncated STAT6 (123-632)-avi purified from E. coli, 1× Streptavidin-terbium (CisBio) prepared by mixing SA-Tb in PPI detection buffer (CisBio), 20 nM proprietary fluorescein-labeled probe, and test compounds in assay buffer consisting of 50 mM HEPES-Na pH 7.5, 100 mM NaCl, 1 mM EDTA, 2 mM DTT, 0.1% Tween-20 with a final volume of 20 uL. Compound stocks were dissolved at 10 mM in 100% DMSO and 11 point titration with 3 fold serial dilution was performed in white, opaque 384 well microplates. Reaction plates were incubated at room temperature for 30 minutes. Plates were centrifuged at low rpm for 5 mins, and the ratio of fluorescence intensities were measured at emission wavelengths for fluorescein acceptor (520 nm) and terbium donor (495 nm) on Envision Plate reader. % Inhibition was calculated from the 520/495 ratio generated by using proprietary positive control compound for 100% inhibition and DMSO only reactions for 0% inhibition. Data was processed and dose response curves were generated using GraphPad Prism to determine the concentration required for inhibiting 50% of the HTRF signal (IC₅₀).

The STAT6 HTRF results are shown in Table 7. The letter codes for IC₅₀ (μM) include: A (<0.1 μM); B (0.1-1 μM); C (>1-10 μM); D (>10-100 μM); and E (>100 μM or not tested).

TABLE 7
STAT6 HTRF Results

| | STAT6 HTRF: |
|---|---|
| | Average IC₅₀ |
| I-# | (μM) |
| I-6 | D |
| I-7 | D |
| I-121 | E |
| I-131 | D |
| I-164 | C |
| I-183 | D |
| I-211 | D |
| I-218 | D |
| I-223 | D |
| I-230 | E |
| I-238 | D |
| I-240 | C |
| I-242 | D |
| I-250 | D |
| I-253 | C |
| I-258 | D |
| I-260 | C |
| I-262 | D |
| I-288 | C |
| I-303 | D |
| I-320 | D |
| I-836 | C |
| I-1102 | B |
| I-1240 | C |
| I-1246 | C |
| I-1260 | D |
| I-1271 | D |
| I-1272 | D |
| I-1273 | D |
| I-1293 | D |
| I-1294 | D |
| I-1295 | D |
| I-1296 | D |
| I-1297 | D |
| I-1298 | D |
| I-1323 | B |
| I-1325 | D |
| I-1583 | C |
| I-1599 | B |
| I-1625 | C |

### Example 2. Phosphorylated STAT6 (pSTAT6) Flow Cytometry Assay in Human PBMC

Phosphorylation of STAT6 in human PBMC was quantitatively measured using flow cytometry technology. Human PBMC were seeded in 96-well plates with a density of 0.3 to 0.5 million cells per well in 100 μl fresh media. Compounds were then added to the assay plates with a final top concentration of up to 30 M in a 1:3 dilution series with total of 9 doses. The assay plates were incubated for 1 hour at 37° C. under 5% CO₂. The cells were then stimulated with 1 ng/ml of IL-4 (R&D Systems, Cat #6507-IL) for 15 minutes, followed by fixation for 30 minutes at 4° C. Cells were spun down at 350 g for 5 minutes. The supernatant was removed and cells were washed once with FACS buffer. Cells were permeabilized with 90% MeOH/H₂O for 30 minutes at 4° C., followed by one wash with FACS buffer. Non-specific binding were blocked with human Fc for 1 hour at 4° C., followed by one wash with FACS buffer. AF488 conjugated pSTAT6 antibody (BD Biosciences, Cat #612600, 1:100 dilution) and PE conjugated CD19 antibody (BD Biosciences, Cat #341103, 1:50 dilution) diluted in 100 μl FACS buffer was added to each well with overnight incubation at 4° C. Cells were then washed once with FACS buffer and resuspended in 200 μl of FACS buffer for flow cytometry to quantify pSTAT6 in CD19+ B cells. The data was analyzed by GraphPad Prism and the dose dependent pSTAT6 inhibition was fit using the four-parameter inhibitor vs. response nonlinear regression.

The STAT6 IL4 pSTAT6 results are shown in Table 8. The letter codes for IC₅₀ (μM) include: A (<0.1 μM); B (0.1-1 μM); C (>1-10 μM); D (>10-100 μM); and E (>100 μM or not tested).

TABLE 8
STAT6 IL4 pSTAT6 Results

| | STAT6 IL4 |
|---|---|
| | pSTAT6 assay |
| | in PBMC: |
| | Average IC₅₀ |
| I-# | (μM) |
| I-6 | D |
| I-7 | E |
| I-121 | E |
| I-131 | E |
| I-164 | E |
| I-183 | E |
| I-211 | E |
| I-218 | E |
| I-223 | E |
| I-230 | E |
| I-238 | E |
| I-240 | E |
| I-242 | E |
| I-250 | E |
| I-253 | E |
| I-258 | E |
| I-260 | E |
| I-262 | E |
| I-288 | E |
| I-303 | E |
| I-320 | E |

### Example 3. Phosphorylated STAT6 (pSTAT6) Flow Cytometry Assay in Human A549 Cells

Phosphorylation of STAT6 in human A549 cells was quantitatively measured using flow cytometry technology.

The STAT6 IL4 pSTAT6 results are shown in Table 9. The letter codes for IC₅₀ (μM) include: A (<0.1 μM); B (0.1-1 μM); C (>1-10 μM); D (>10-100 μM); and E (>100 μM or not tested).

TABLE 9
STAT6 IL4 pSTAT6 Results.

| | STAT6 IL4 |
|---|---|
| | pSTAT6 assay |
| | in A549 cells: |
| | Average IC₅₀ |
| I-# | (μM) |
| I-836 | C |
| I-1102 | B |

1. An inhibitor compound of formula I-a: 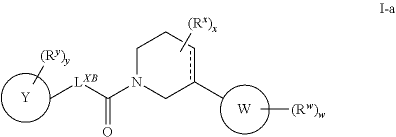
or a pharmaceutically acceptable salt thereof, wherein: [symbol]
is a single or double bond;
Ring W is phenyl, a 5- to 6-membered monocyclic heteroaryl having 1-3 heteroatoms independently selected from nitrogen, oxygen, or sulfur, a 9-membered bicyclic saturated heterocyclyl having 1-3 heteroatoms independently selected from nitrogen, oxygen, or sulfur, 10-membered bicyclic saturated or partially unsaturated heterocyclyl having 1-3 heteroatoms independently selected from nitrogen, oxygen, or sulfur, naphthyl, a 10-membered bicyclic heteroaryl having 1-3 heteroatoms independently selected from nitrogen, oxygen, or sulfur, or a 13-membered tricyclic heteroaryl having 1-3 heteroatoms independently selected from nitrogen, oxygen, or sulfur;
each R^{w} is independently selected from halogen, —CN, —OR^{w1}, —N(R^{w1})₂, —(CH₂)ₙ C(O)NR₂, —(CH₂)ₙ N(R)C(O)NR₂, —(O)ₘ-phenyl, C₁₋₄ aliphatic, a 6-membered monocyclic saturated or partially unsaturated heterocyclyl having 1-3 heteroatoms independently selected from nitrogen, oxygen, or sulfur, or a 5- to 6-membered monocyclic heteroaryl having 1-3 heteroatoms independently selected from nitrogen, oxygen, or sulfur, wherein each phenyl, heterocyclyl, or heteroaryl is optionally substituted with halogen, C₁₋₄ aliphatic, —OR, or —C(O)NR₂;
each R^{w1} is independently selected from hydrogen, C₁₋₄ aliphatic, phenyl, or 6-membered monocyclic heteroaryl having 1-3 heteroatoms independently selected from nitrogen, oxygen, or sulfur, wherein each aliphatic, phenyl, or heteroaryl is optionally substituted with halogen, —OR, or —C(O)OR;
each R^{x} is independently C₁₋₄ aliphatic, halogen, or —C(O)NR₂;
each R is independently hydrogen or C₁₋₄ aliphatic;
L^{XB} is a bivalent straight or branched C₁₋₃ aliphatic wherein one methylene unit is optionally replaced with —NR—;
Ring Y is a 5-membered monocyclic heteroaryl having 1-3 heteroatoms independently selected from nitrogen, oxygen, or sulfur, phenyl, or a 10-membered bicyclic saturated or partially unsaturated heterocyclyl having 1-3 heteroatoms independently selected from nitrogen, oxygen, or sulfur;
each R^{y} is independently C₁₋₄ aliphatic, halogen, —OR, or —C(O)NR₂;
each m is independently 0 or 1;
each n is independently 0, 1, 2, or 3; and
each w, x, and y is independently 0, 1, 2, 3, or 4.

2. An inhibitor compound of formula I-b: 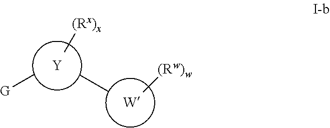
Ring W′ is 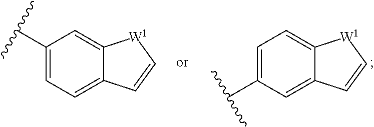
W¹ is O, NH, or NR^{w};
Ring X is a 5-membered monocyclic heteroarylenyl having 1-3 heteroatoms independently selected from nitrogen, oxygen, or sulfur, phenylenyl, or a 5- or 7-membered saturated or partially unsaturated monocyclic heterocyclylenyl having 1-3 heteroatoms independently selected from nitrogen, oxygen, or sulfur;
each R^{w} is independently selected from halogen, —CN, —OR^{w1}, —N(R^{w1})₂, —(CH₂)ₙ C(O)NR₂, —(CH₂)ₙ N(R)C(O)NR₂, —(O)ₘ-phenyl, C₁₋₄ aliphatic, a 6-membered saturated or partially unsaturated monocyclic heterocyclyl having 1-3 heteroatoms independently selected from nitrogen, oxygen, or sulfur, or a 5- to 6-membered monocyclic heteroaryl having 1-3 heteroatoms independently selected from nitrogen, oxygen, or sulfur, wherein each phenyl, heterocyclyl, or heteroaryl is optionally substituted with halogen, C₁₋₄ aliphatic, —OR, or —C(O)NR₂;
each R^{w1} is independently selected from hydrogen, C₁₋₄ aliphatic, phenyl, or 6-membered monocyclic heteroaryl having 1-3 heteroatoms independently selected from nitrogen, oxygen, or sulfur, wherein each aliphatic, phenyl, or heteroaryl is optionally substituted with halogen, —OR, or —C(O)OR;
each R^{x} is independently C₁₋₄ aliphatic, halogen, or —C(O)NR₂;
each R is independently hydrogen or C₁₋₄ aliphatic;
G is hydrogen or 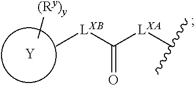
L^{XA} is a covalent bond or a bivalent straight or branched C₁₋₃ aliphatic;
L^{XB} is a bivalent straight or branched C₁₋₃ aliphatic wherein one methylene unit is optionally replaced with —NR—;
Ring Y is a 5-membered monocyclic heteroaryl having 1-3 heteroatoms independently selected from nitrogen, oxygen, or sulfur, phenyl, or a 10-membered bicyclic saturated or partially unsaturated heterocyclyl having 1-3 heteroatoms independently selected from nitrogen, oxygen, or sulfur;
each R^{y} is independently C₁₋₄ aliphatic, halogen, —OR, or —C(O)NR₂;
each m is independently 0 or 1;
each n is independently 0, 1, 2, or 3; and
each w, x, and y is independently 0, 1, 2, 3, or 4.

3. The inhibitor compound of claim 1, wherein Ring W is:

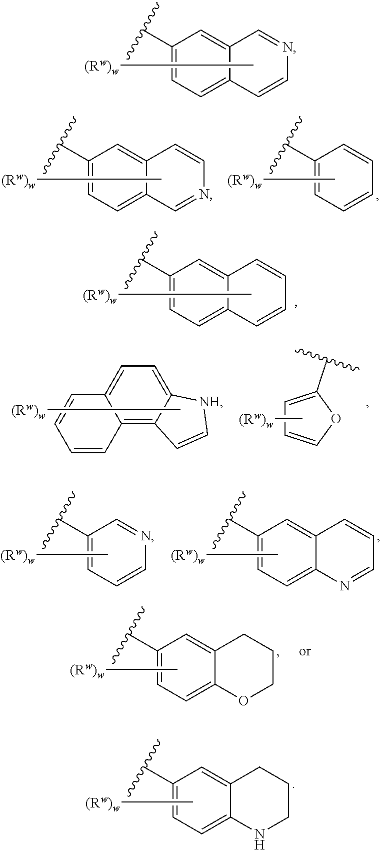

4. The inhibitor compound of claim 2, wherein W¹ is O.

5. The inhibitor compound of claim 2, wherein W¹ is —NH— or —NR^{w}—.

6. The inhibitor compound of claim 2, wherein Ring X is: 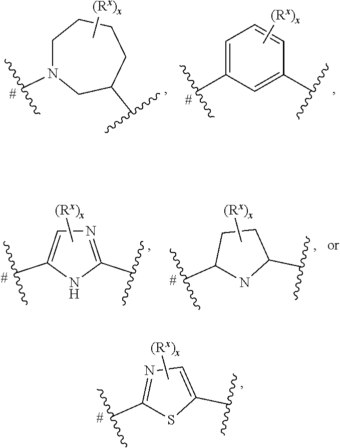
wherein # represents the point of attachment to G.

7. The inhibitor compound of claim 2, wherein G is hydrogen.

8. The inhibitor compound of claim 2, wherein G is

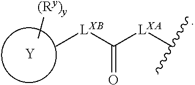

9. The inhibitor compound of claim 1, wherein Ring Y is:

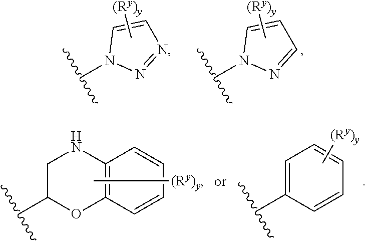

10. The inhibitor compound of claim 1, wherein an occurrence of R^{w} is —(O)ₘ-phenyl, wherein the phenyl is optionally substituted with halogen, —OR, or —C(O)NR₂.

11-15. (canceled)

16. The inhibitor compound of claim 1, wherein the inhibitor compound is of formulae II-a, III-a, or IV-a: 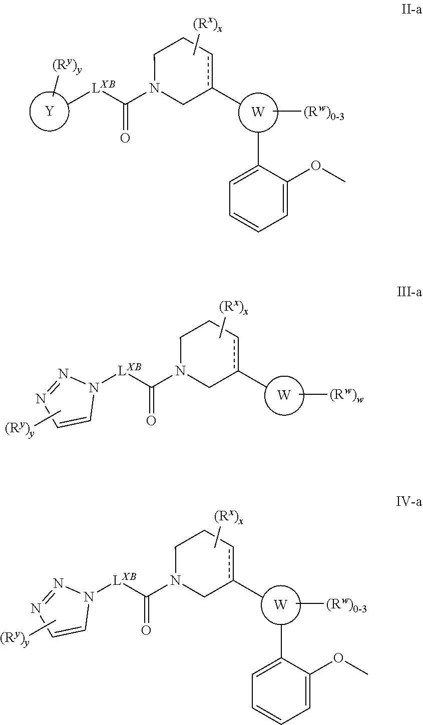
or a pharmaceutically acceptable salt thereof.

17. The inhibitor compound of claim 2, wherein the inhibitor compound is of formula I-b-1 or 1-b-2: 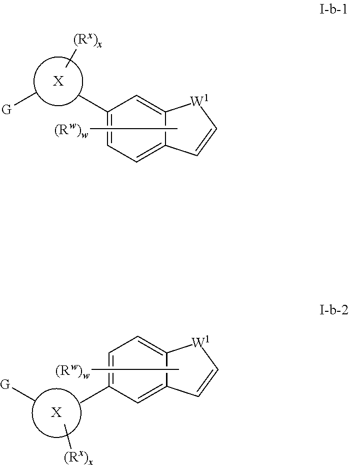
or a pharmaceutically acceptable salt thereof.

18. (canceled)

19. The inhibitor compound of claim 2, wherein the inhibitor compound is of formulae II-b, III-b, IV-b, V-b, VI-b, or VII-b: 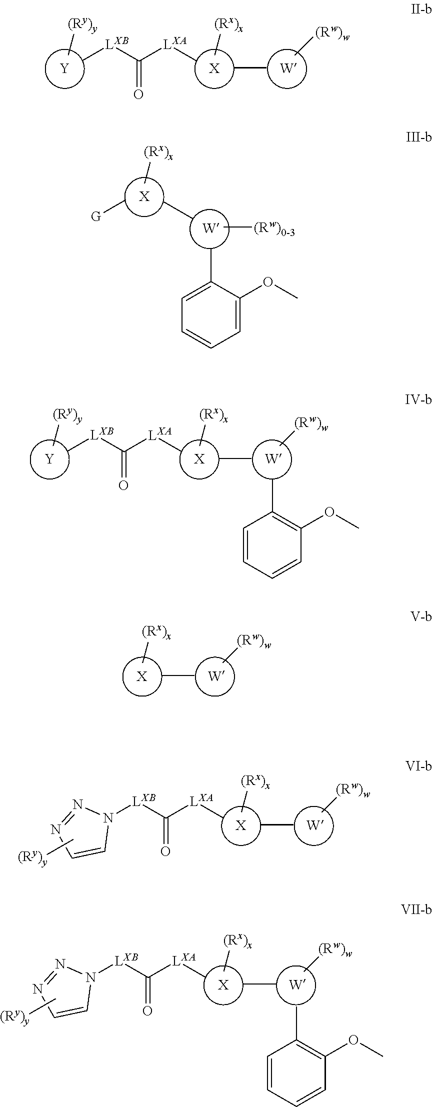
or a pharmaceutically acceptable salt thereof.

20. A compound selected from any one of the compounds depicted in Table 1A, Table 1B, Table 2A, or Table 2B, or a pharmaceutically acceptable salt thereof.

21. A pharmaceutical composition comprising an inhibitor compound of claim 1, and a pharmaceutically acceptable carrier, adjuvant, or vehicle.

22. A method of inhibiting STAT6, the method comprising contacting STAT6 with an inhibitor compound of claim 1, or a pharmaceutically acceptable salt thereof.

23. The method of claim 22, wherein the contacting occurs in a patient or biological sample.

24. A pharmaceutical composition comprising an inhibitor compound of claim 2, and a pharmaceutically acceptable carrier, adjuvant, or vehicle.

25. A method of inhibiting STAT6, the method comprising contacting STAT6 with an inhibitor compound of claim 2, or a pharmaceutically acceptable salt thereof.

26. The method of claim 25, wherein the contacting occurs in a patient or biological sample.
